(19) United States
(12) Patent Application Publication
(10) Pub. No.: US 2021/0022662 A1
(43) Pub. Date: Jan. 28, 2021

(54) ELECTRONIC DEVICE AND METHOD FOR PROVIDING CONTENT ACCORDING TO SKIN TYPE OF USER

(71) Applicant: SAMSUNG ELECTRONICS CO., LTD., Suwon-si (KR)

(72) Inventors: Dae-Young HYUN, Suwon-si, Gyeonggi-do (KR); Sun-Ah KIM, Seongnam-si, Gyeonggi-do (KR); Jae-Jin KIM, Ansan-si, Gyeonggi-do (KR); Ji-Yun KIM, Suwon-si, Gyeonggi-do (KR); Hong-II KIM, Yongin-si, Gyeonggi-do (KR); Joo-Young SON, Yongin-si, Gyeonggi-do (KR); Kyung-Ho JEONG, Seoul (KR); Ga-Hyun JOO, Suwon-si, Gyeonggi-do (KR); Tae-Hwa HONG, Seoul (KR)

(73) Assignee: SAMSUNG ELECTRONICS CO., LTD, Suwon-si (KR)

(21) Appl. No.: 15/999,065

(22) PCT Filed: Feb. 17, 2017
(86) PCT No.: PCT/KR2017/001812

§ 371 Date: Aug. 17, 2018

(30) Foreign Application Priority Data
Feb. 17, 2016 (KR) 10-2016-0018749

(51) Int. Cl. A61B 5/00

(52) U.S. Cl. CPC A61B 5/441; A61B 5/7435; A61B 5/743; A61B 5/0077

(57) ABSTRACT

The present invention provides an electronic device and a method for providing content according to the skin type of a user. The electronic devices according to various embodiments comprise: a display; and a processor, wherein the processor obtains skin diagnosis information which represents the skin condition of a user, determines the skin type of the user on the basis of skin diagnosis information, and displays, on the display, information on the determined skin type and content corresponding to the determined skin type. Other embodiments are also available.

### CROSS-REFERENCE TO RELATED APPLICATION(S)

This application is a National Phase Entry of PCT International Application No. PCT/KR2017/001812, which was filed on Feb. 17, 2017, and claims priority to Korean Patent Application No. 10-2016-0018749, which was filed on Feb. 17, 2016, the contents of which are incorporated herein by reference.

### BACKGROUND

### 1. Field

The various embodiments of the present disclosure are intended to provide content related to the skin of a user.

### 2. Description of the Related Art

In general, as interest in skin care grows, a variety of skin care products are emerging. In particular, cosmetics are classified into various detailed categories depending on the stages of makeup, the skin types, and focused functions of cosmetics. Human skin may also be divided into skin types according to the states of oil, moisture, pigmentation, trouble, pores, roughness, and the like. Accordingly, a desire for exactly knowing one's skin condition is steadily growing in order to select a skin care product and a skin care method suitable therefor.

### SUMMARY

Recently, there has been a growing interest in a method for identifying the skin condition of a user and providing a variety of content related thereto.

Accordingly, a method for finely measuring the skin type of a user on the basis of a mobile device and providing a customized skin care service for the skin type.

Various embodiments of the present disclosure provide an electronic device and a method for providing content according to the skin type of a user.

In order to realize the above mention, an electronic device, according to various embodiments of the present disclosure, may include: a display; and a processor, wherein the processor may be configured to: obtain skin diagnosis information indicating the skin condition of a user; based on the skin diagnosis information, determine the skin type of the user; display information on the determined skin type and content corresponding to the determined skin type on the display.

In order to realize the above mention, according to various embodiments of the present disclosure, a method for providing content according to a skin type in an electronic device may include: obtaining skin diagnosis information indicating the skin condition of a user; based on the skin diagnosis information, determining the skin type of the user; and displaying information on the determined skin type and content corresponding to the determined skin type on a display

Various embodiments of the present disclosure can provide an electronic device for providing content depending on mobile-based skin type measurement, thereby allowing a user to conveniently measure the skin type according to skin diagnosis and to easily receive content related to the measured skin type.

In various embodiments of the present disclosure, it is possible to finely measure the skin type of a user, thereby providing customized skin care services according thereto.

### BRIEF DESCRIPTION OF THE DRAWINGS

FIG. 1 illustrates a network environment including an electronic device according to various embodiments.

FIG. 2 illustrates a block diagram of an electronic device according to various embodiments.

FIG. 3 illustrates a block diagram of a program module according to various embodiments.

FIG. 4 illustrates a configuration diagram of a system for providing content according to a skin type according to various embodiments.

FIG. 5 illustrates a configuration diagram of an electronic device according to various embodiments.

FIG. 6 illustrates a configuration diagram of a server according to various embodiments.

FIG. 7 illustrates a configuration diagram of a service provider device according to various embodiments.

FIG. 8 illustrates a flowchart showing a process for providing content related to a skin type in an electronic device according to various embodiments.

FIG. 9 illustrates a flowchart in which an electronic device, based on skin diagnosis information and context information, provides content related to a skin type according to various embodiments.

FIG. 10 illustrates a flowchart showing a process for determining a skin type using a user interface to receive, from a user, replies to queries predetermined for skin diagnosis according to various embodiments.

FIG. 11 illustrates a flowchart showing a process in which an electronic device determines a skin type using a camera module according to various embodiments.

FIG. 12 illustrates a flowchart showing a process in which an electronic device determines a skin type using a user interface for determining the skin type of a user and a camera module or a sensor module according to various embodiments.

FIG. 13 illustrates a flowchart showing a process in which an electronic device receives content corresponding to a skin type from a server and provides the same according to various embodiments.

FIG. 14 illustrates a flowchart showing a process in which a server provides content corresponding to a skin type upon request by an electronic device according to various embodiments.

FIG. 15 illustrates a flowchart showing a process in which a service provider device matches at least one skin type with content according to various embodiments.

FIG. 16 illustrates a flowchart showing a process for providing content corresponding to a skin type between an electronic device and a server according to various embodiments.

FIG. 17 illustrates a flowchart showing a process of matching content corresponding to a skin type between a server and a service provider device according to various embodiments.

FIG. 18 illustrates an exemplary view showing a skin type according to various embodiments.

FIG. 19 illustrates exemplary views explaining an operation of selecting ratings for respective skin analysis items depending on a service provider according to various embodiments.

FIG. 20 illustrates exemplary views explaining skin rating information according to various embodiments.

FIGS. 21 to 36 illustrate exemplary views explaining the operation of a skin care application to provide content for each skin type in an electronic device according to various embodiments.

FIG. 37 illustrates an exemplary view of a user interface for selecting content according to a skin type in a service provider device according to various embodiments.

### DETAILED DESCRIPTION

Hereinafter, various embodiments of the present disclosure will be described with reference to the accompanying drawings. However, it should be understood that there is no intent to limit the present disclosure to the particular forms disclosed herein; rather, the present disclosure should be construed to cover various modifications, equivalents, and/or alternatives of embodiments of the present disclosure. In describing the drawings, similar reference numerals may be used to designate similar constituent elements.

As used herein, the expression “have”, “may have”, “include”, or “may include” refers to the existence of a corresponding feature (e.g., numeral, function, operation, or constituent element such as component), and does not exclude one or more additional features.

In the present disclosure, the expression “A or B”, “at least one of A or/and B”, or “one or more of A or/and B” may include all possible combinations of the items listed. For example, the expression “A or B”, “at least one of A and B”, or “at least one of A or B” refers to all of (1) including at least one A, (2) including at least one B, or (3) including all of at least one A and at least one B.

The expression “a first”, “a second”, “the first”, or “the second” used in various embodiments of the present disclosure may modify various components regardless of the order and/or the importance but does not limit the corresponding components. For example, a first user device and a second user device indicate different user devices although both of them are user devices. For example, a first element may be termed a second element, and similarly, a second element may be termed a first element without departing from the scope of the present disclosure.

It should be understood that when an element (e.g., first element) is referred to as being (operatively or communicatively) “connected,” or “coupled,” to another element (e.g., second element), it may be directly connected or coupled directly to the other element or any other element (e.g., third element) may be interposer between them. In contrast, it may be understood that when an element (e.g., first element) is referred to as being “directly connected,” or “directly coupled” to another element (second element), there are no element (e.g., third element) interposed between them.

The expression “configured to” used in the present disclosure may be exchanged with, for example, “suitable for”, “having the capacity to”, “designed to”, “adapted to”, “made to”, or “capable of” according to the situation. The term “configured to” may not necessarily imply “specifically designed to” in hardware. Alternatively, in some situations, the expression “device configured to” may mean that the device, together with other devices or components, “is able to”. For example, the phrase “processor adapted (or configured) to perform A, B, and C” may mean a dedicated processor (e.g., embedded processor) only for performing the corresponding operations or a generic-purpose processor (e.g., Central Processing Unit (CPU) or Application Processor (AP)) that can perform the corresponding operations by executing one or more software programs stored in a memory device.

The terms used in the present disclosure are only used to describe specific embodiments, and are not intended to limit the present disclosure. A singular expression may include a plural expression unless they are definitely different in a context. Unless defined otherwise, all terms used herein, including technical and scientific terms, have the same meaning as those commonly understood by a person skilled in the art to which the present disclosure pertains. Such terms as those defined in a generally used dictionary may be interpreted to have the meanings equal to the contextual meanings in the relevant field of art, and are not to be interpreted to have ideal or excessively formal meanings unless clearly defined in the present disclosure. In some cases, even the term defined in the present disclosure should not be interpreted to exclude embodiments of the present disclosure.

An electronic device according to various embodiments of the present disclosure may include at least one of, for example, a smart phone, a tablet Personal Computer (PC), a mobile phone, a video phone, an electronic book reader (e-book reader), a desktop PC, a laptop PC, a netbook computer, a workstation, a server, a Personal Digital Assistant (PDA), a Portable Multimedia Player (PMP), a MPEG-1 audio layer-3 (MP3) player, a mobile medical device, a camera, and a wearable device. According to various embodiments, the wearable device may include at least one of an accessory type (e.g., a watch, a ring, a bracelet, an anklet, a necklace, a glasses, a contact lens, or a Head-Mounted Device (HMD)), a fabric or clothing integrated type (e.g., an electronic clothing), a body-mounted type (e.g., a skin pad, or tattoo), and a bio-implantable type (e.g., an implantable circuit).

According to some embodiments, the electronic device may be a home appliance. The home appliance may include at least one of, for example, a television, a Digital Video Disk (DVD) player, an audio, a refrigerator, an air conditioner, a vacuum cleaner, an oven, a microwave oven, a washing machine, an air cleaner, a set-top box, a home automation control panel, a security control panel, a TV box (e.g., Samsung HomeSync™, Apple TV™, or Google TV™), a game console (e.g., Xbox™ and PlayStation™), an electronic dictionary, an electronic key, a camcorder, and an electronic photo frame.

According to another embodiment, the electronic device may include at least one of various medical devices (e.g., various portable medical measuring devices (a blood glucose monitoring device, a heart rate monitoring device, a blood pressure measuring device, a body temperature measuring device, etc.), a Magnetic Resonance Angiography (MRA), a Magnetic Resonance Imaging (MRI), a Computed Tomography (CT) machine, and an ultrasonic machine), a navigation device, a Global Positioning System (GPS) receiver, an Event Data Recorder (EDR) , a Flight Data Recorder (FDR) , a Vehicle Infotainment Devices, an electronic devices for a ship (e.g., a navigation device for a ship, and a gyro-compass), avionics, security devices, an automotive head unit, a robot for home or industry, an Automatic Teller's Machine (ATM) in banks, Point Of Sales (POS) in a shop, or internet device of things (e.g., a light bulb, various sensors, electric or gas meter, a sprinkler device, a fire alarm, a thermostat, a streetlamp, a toaster, a sporting goods, a hot water tank, a heater, a boiler, etc.).

According to some embodiments, the electronic device may include at least one of a part of furniture or a building/structure, an electronic board, an electronic signature receiving device, a projector, and various kinds of measuring instruments (e.g., a water meter, an electric meter, a gas meter, and a radio wave meter). The electronic device according to various embodiments of the present disclosure may be a combination of one or more of the aforementioned various devices. The electronic device according to some embodiments of the present disclosure may be a flexible device. Further, the electronic device according to an embodiment of the present disclosure is not limited to the aforementioned devices, and may include a new electronic device according to the development of technology.

Hereinafter, an electronic device according to various embodiments of the present disclosure will be described with reference to the accompanying drawings. In the present disclosure, the term “user” may indicate a person using an electronic device or a device (e.g., an artificial intelligence electronic device) using an electronic device.

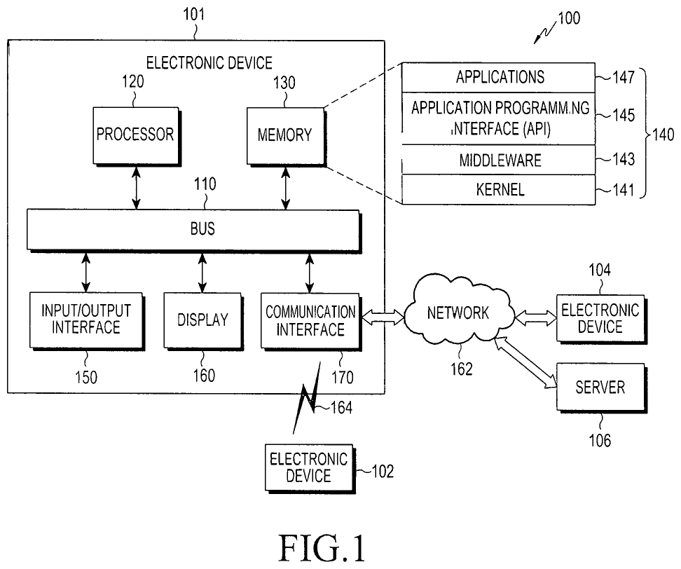

Referring to FIG. 1, an electronic device 101 in a network environment 100 is disclosed in various embodiments. The electronic device 101 may include a bus 110, a processor 120, a memory 130, an input/output interface 150, a display 160, and a communication interface 170. In some embodiments, the electronic device 101 may exclude at least one of the above elements, or may further include other elements in addition thereto.

The bus 110, for example, may include a circuit for connecting the elements 110 to 170 with each other and transferring communication signal(e.g., control messages and/or data) between the elements.

The processor 120 may include at least one of a central processing unit (CPU), an application processor (AP), or a communication processor (CP). The processor 120, for example, may perform a calculation or data processing in relation to control and/or communication of one or more other elements of the electronic device 101. The processor 120 may be referred to as a “controller”, may include the controller as a part thereof, or may constitute the controller.

The memory 130 may include a volatile and/or non-volatile memory. For example, the memory 130 may store commands or data in relation to one or more other elements of the electronic device 101. According to an embodiment, the memory 130 may store software and/or programs 140. For example, the programs 140 may include a kernel 141, middleware 143, an application programming interface (API) 145, and/or application programs (or “applications”) 147. At least some of the kernel 141, the middleware 143, or the API 145 may be referred to as an “operating system (OS)”.

The kernel 141, for example, may control or manage system resources (e.g., the bus 110, the processor 120, the memory 130, or the like), which are used to execute operations or functions that are implemented in other programs (e.g., the middleware 143, the API 145, or the application programs 147). In addition, the kernel 141 may provide an interface by which the middleware 143, the API 145, or the application programs 147 may access respective elements of the electronic device 101, thereby controlling or managing system resources.

The middleware 143, for example, may play the intermediate role between the API 145 or the application programs 147 and the kernel 141 to communicate with each other for transmission and reception of data.

The middleware 143 may process one or more operation requests received from the application programs 147 according to priority. For example, the middleware 143 may give priority for using the system resources (e.g., the bus 110, the processor 120, the memory 130, or the like) of the electronic device 101 to at least one of the application programs 147. For example, the middleware 143 may perform scheduling or load balancing for one or more operation requests by processing the one or more operation requests according to the priority given to at least one of the application programs.

The API 145, for example, may be an interface by which the applications 147 control functions provided by the kernel 141 or the middleware 143. For example, the API 145 may include one or more interfaces or functions (e.g., instructions) for file control, window control, image processing, text control, or the like.

The input/output interface 150, for example, may play the role of an interface that transfers commands or data received from a user or other external devices to other elements of the electronic device 101. In addition, the input/output interface 150 may output commands or data received from the other elements of the electronic device 101 to the user or other external devices.

The display 160, for example, may include a liquid crystal display (LCD), a light-emitting diode (LED) display, an organic light-emitting diode (OLED) display, a microelectromechanical systems (MEMS) display, or an electronic paper display. For example, the display 160 may display a variety of content (e.g., text, images, videos, icons, symbols, or the like) to the user. The display 160 may include a touch screen, and may receive, for example, touch input, gesture input, proximity input, or hovering input using electronic pens or a user's body part.

The communication interface 170, for example, may establish communication between the electronic device 101 and external devices (e.g., a first external electronic device 102, a second external electronic device 104, or a server 106). For example, the communication interface 170 may be connected to the network 162 through wireless communication or wired communication, thereby communicating with external devices (e.g., the second external electronic device 104 or the server 106).

For example, the wireless communication may use, as a cellular communication protocol, at least one of LTE (Long-Term Evolution), LTE-A (LTE Advance), CDMA (Code Division Multiple Access), WCDMA (Wideband CDMA), a UMTS (Universal Mobile Telecommunications System), WiBro (Wireless Broadband), a GSM (Global System for Mobile Communications), or the like. In addition, the wireless communication, for example, may include a short-range communication 164. The short-range communication 164, for example, may include at least one of Wi-Fi (Wireless Fidelity), Bluetooth, NFC (Near Field Communication), or a GNSS (Global Navigation Satellite System). The GNSS, for example, may include at least one of a GPS (Global Positioning System), a GLONASS (Global Navigation Satellite System), the Beidou Navigation Satellite System (hereinafter, “Beidou”), Galileo, or the European global satellite-based navigation system according to the usage area or bandwidth. Hereinafter, “GPS” may be used interchangeably with “GNSS” in the present specification. For example, the wired communication may include at least one of a USB (Universal Serial Bus), an HDMI (High-Definition Multimedia Interface), RS-232 (Recommended Standard 232), or a POTS (Plain Old Telephone Service). The network 162 may include at least one of the telecommunication networks, such as a computer network (e.g., LAN or WAN), the Internet, or a telephone network.

The first external device 102 and the second external device 104 may be the same as, or different from, the electronic device 101 as to the type thereof.

According to an embodiment, the server 106 may include a group of one or more servers.

According to various embodiments, at least some or all of the operations that are executed in the electronic device 101 may be executed by one or more other electronic devices (e.g., the electronic device 102 or 104, or the server 106).

According to an embodiment, in the case where the electronic device 101 executes specific functions or services automatically or upon request, the electronic device 101 may make a request to other devices (e.g., the electronic device 102 or 104, or the server 106) for at least some of the functions related to the same, instead of executing the functions or services by itself, or in addition thereto. Other electronic devices (e.g., the electronic device 102 or 104, or the server 106) may execute the requested functions or additional functions, and may transfer the result thereof to the electronic device 101. The electronic device 101 may provide the requested functions or services by providing the received result without further processing or by additionally processing the same. To this end, for example, cloud computing, distributed computing, or client-server computing technology may be used.

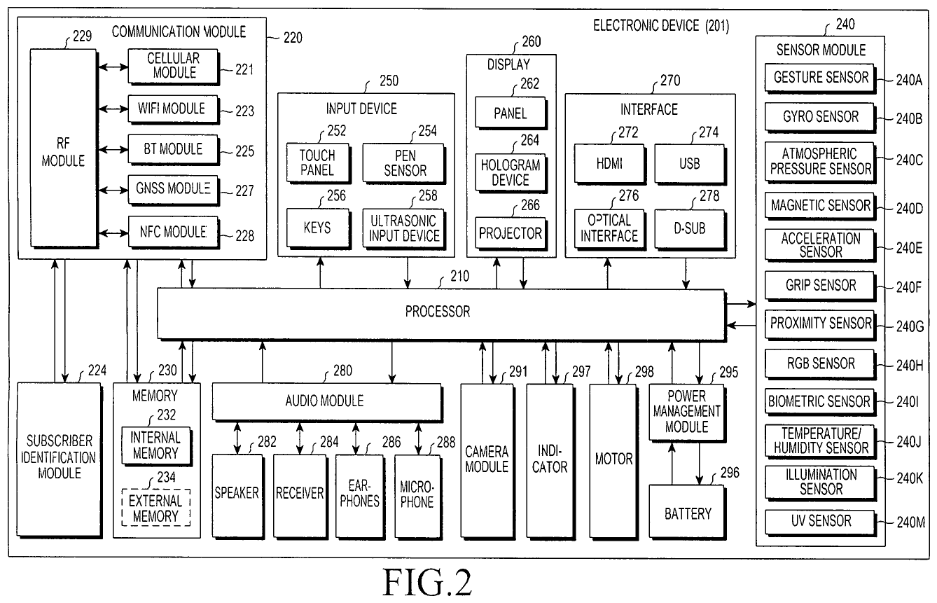

FIG. 2. is a block diagram of an electronic device according to various embodiments. An electronic device 201, for example, may include all or some of the elements of the electronic device 101 shown in FIG. 1. The electronic device 201 may include one or more processors {e.g., application processors (AP)} 210, a communication module 220, a subscriber identification module 224, a memory 230, a sensor module 240, an input device 250, a display 260, an interface 270, an audio module 280, a camera module 291, a power management module 295, a battery 296, an indicator 297, and a motor 298.

The processor 210, for example, may control a multitude of hardware or software elements connected with the processor 210, and may perform processing of a variety of data and a calculation by executing an operating system or application programs. The processor 210 may be implemented by, for example, a system on chip (SoC). According to an embodiment, the processor 210 may further include a graphic processing unit (GPU) and/or an image signal processor. The processor 210 may include at least some (e.g., the cellular module 221) of the elements shown in FIG. 2. The processor 210 may load commands or data received from one or more other elements (e.g., a non-volatile memory) to a volatile memory to then process the same, and may store a variety of data in a non-volatile memory.

According to an embodiment, the processor 210 may determine the skin type of a user, and may provide the content corresponding to the determined skin type. The skin type may be determined based on skin diagnosis information indicating the skin condition of the user, or may be determined based on the skin diagnosis information and context information indicating the health condition or environment state of the user. The skin diagnosis information may include: first skin analysis information obtained by analyzing replies, which are received from a user, to queries predetermined for skin diagnosis according to skin analysis items including at least one of oil, moisture, wrinkles, pigmentation, trouble, or the like; and second skin analysis information obtained by analyzing the skin of a user according to the skin analysis items using a camera module or a sensor module. The context information may include environment information containing at least one of an ultraviolet (UV) index, temperature, humidity, and fine dust concentration corresponding to a user's surrounding environment. The context information may further include health information of the user in addition to the environment information of the user. The health information may be information (e.g., health analysis information, health analysis values, health characteristic information, health characteristic values, or the like) obtained by analyzing the user's health condition according to health analysis items including at least one of the amount of calorie consumption, the amount of moisture intake, sleeping time, or stress of the user. The environment information may be information (e.g., environment analysis information, environment analysis values, environment characteristic information, environment characteristic values, or the like) obtained by analyzing the surrounding environment of the user according to environment analysis items including at least one of a UV index, temperature, humidity, fine dust concentration, and the like. The environment information may be provided from an environment information provider such as the National Weather Service.

The processor 210 may retrieve and provide the content corresponding to the determined skin type, or may make a request to a server (e.g., the server 106) for the content corresponding to the skin type, and may provide the content received from the server (e.g., the server 106).

The communication module 220 may have a configuration the same as or similar to that of the communication interface 170 of FIG. 1. The communication module 220, for example, may include a cellular module 221, a Wi-Fi module 223, a Bluetooth module 225, a GNSS module 227 (e.g., a GPS module, a Glonass module, the Beidou module, or the Galileo module), an NFC module 228, and a radio frequency (RF) module 779

According to an embodiment, the communication module 220 may transmit a request message for requesting the content corresponding to the determined skin type to the server (e.g., the server 106), or may receive the content corresponding to the determined skin type from the server (e.g., the server 106).

The cellular module 221, for example, may provide services of voice calls, video calls, text messaging, or the Internet through communication networks. According to an embodiment, the cellular module 221 may perform identification and verification of the electronic device 201 in communication networks using the subscriber identification module (e.g., a SIM card) 224. According to an embodiment, the cellular module 221 may perform at least some of the functions provided by the processor 210. According to an embodiment, the cellular module 221 may include a communication processor (CP).

For example, each of the Wi-Fi module 223, the Bluetooth module 225, the GNSS module 227, and the NFC module 228 may include a processor for processing data transmitted and received through a corresponding module. According to some embodiments, at least some (e.g., two or more) of the cellular module 221, the Wi-Fi module 223, the Bluetooth module 225, the GNSS module 227, or the NFC module 228 may be included in a single integrated chip (IC) or IC package.

The RF module 229, for example, may transmit and receive communication signals (e.g., RF signals). The RF module 229 may include, for example, a transceiver, a power amp module (PAM), a frequency filter, a low-noise amplifier (LNA), antennas, or the like. According to another embodiment, at least one of the cellular module 221, the Wi-Fi module 223, the Bluetooth module 225, the GNSS module 227, or the NFC module 228 may transmit and receive RF signals through a separate RF module.

The subscriber identification module 224, for example, may include a card including a subscriber identification module and/or an embedded SIM, and may contain inherent identification information {e.g., an integrated circuit card identifier (ICCID)} or subscriber information {e.g., an international mobile subscriber identity (IMSI)}.

The memory 230 (e.g., the memory 130), for example, may include an internal memory 232 or an external memory 234. The internal memory 232, for example, may include at least one of volatile memories {e.g., a dynamic RAM (DRAM), a static RAM (SRAM), a synchronous dynamic RAM (SDRAM), or the like} or non-volatile memories {e.g., an one-time programmable ROM (OTPROM), a programmable ROM (PROM), an erasable and programmable ROM (EPROM), an electrically erasable and programmable ROM (EEPROM), a mask ROM, a flash ROM, a flash memory (e.g., NAND flash or NOR flash), a hard drive, a solid state drive (SSD), or the like}.

The external memory 234 may include a flash drive, and may further include, for example, compact flash (CF), secure digital (SD), micro secure digital (Micro-SD), mini secure digital (Mini-SD), extreme digital (xD), a multi-media card (MMC), a memory stick, or the like. The external memory 234 may be functionally and/or physically connected with the electronic device 201 through any of various interfaces.

The sensor module 240, for example, may measure physical quantities or may detect the operation state of the electronic device 201, thereby converting the measured or detected information to electric signals. The sensor module 240 may include at least one of, for example, a gesture sensor 240A, a gyro-sensor 240B, an atmospheric pressure sensor 240C, a magnetic sensor 240D, an acceleration sensor 240E, a grip sensor 240F, a proximity sensor 240G, a color sensor 240H {e.g., a red-green-blue (RGB) sensor}, a biometric sensor 240I, a temperature/humidity sensor 240J, an illuminance sensor 240K, or an ultra-violet (UV) sensor 240M. Additionally or alternatively, the sensor module 240, for example, may further include an E-nose sensor, an electromyography (EMG) sensor, an electroencephalogram (EEG) sensor, an electrocardiogram (ECG) sensor, an infrared (IR) sensor, an iris sensor, and/or a fingerprint sensor. The sensor module 240 may further include a control circuit for controlling one or more sensors included therein. In some embodiments, the electronic device 201 may further include a processor as a part of the processor 210 or separately from the processor 210, which is configured to control the sensor module 240, thereby controlling the sensor module 240 while the processor 210 is in a sleep mode.

According to an embodiment, the sensor module 240 may include at least one of a sensor for sensing moisture or a sensor for sensing facial color.

The input device 250, for example, may include a touch panel 252, a (digital) pen sensor 254, keys 256, or an ultrasonic input device 258.

According to an embodiment, the input device 250 may receive input for receiving the content corresponding to the skin type, or may receive, from the user, replies to queries predetermined for skin diagnosis.

The touch panel 252 may use at least one of, for example, a capacitive type, a pressure-sensitive type, an infrared type, or an ultrasonic type. In addition, the touch panel 252 may further include a control circuit. The touch panel 252 may further include a tactile layer in order to provide a user with a tactile reaction.

For example, the (digital) pen sensor 254 may be a part of the touch panel, or may include a separate recognition sheet. The keys 256 may include, for example, physical buttons, optical keys, or a keypad. The ultrasonic input device 258 may detect ultrasonic waves that are generated in the input means through a microphone (e.g., a microphone 288), thereby identifying data corresponding to the detected ultrasonic waves.

The display 260 (e.g., the display 160) may include a panel 262, a hologram device 264, or a projector 266. The panel 262 may include the same or a similar configuration as the display 160 of FIG. 1. The panel 262 may be implemented to be, for example, flexible, transparent, or wearable. The panel 262 may be configured with the touch panel 252 as a single module. The hologram device 264 may display 3D images in the air using light interference. The projector 266 may display images by projecting light onto a screen. The screen may be positioned, for example, inside or outside the electronic device 201. According to an embodiment, the display 260 may further include a control circuit for controlling the panel 262, the hologram device 264, or the projector 266.

According to an embodiment, the display 260 may display a user interface to receive, from the user, replies to queries predetermined for skin diagnosis. For example, the user interface may include an area for displaying queries predetermined according to skin analysis items including at least one of oil, moisture, wrinkles, pigmentation, trouble, and the like and an area for receiving a user input on the displayed queries.

The interface 270 may include, for example, a high-definition multimedia interface (HDMI) 272, a universal serial bus (UBS) 274, an optical interface 276, or a D-subminiature (D-sub) interface 278. The interface 270 may be included in, for example, the communication interface 170 shown in FIG. 1. Additionally or alternatively, the interface 270 may include, for example, a mobile high-definition link (MHL) interface, a secure digital (SD) card/multi-media card (MMC) interface, or an infrared data association (IrDA) standard interface.

The audio module 280, for example, may convert a sound into an electric signal, and vice versa. At least some elements of the audio module 280 may be included, for example, in the input/output interface 150 shown in FIG. 1. For example, the audio module 280 may process voice information that is input or output through a speaker 282, a receiver 284, earphones 286, or a microphone 288.

The camera module 291, for example, may be a device for photographing still and moving images, and, according to an embodiment, the camera module 291 may include one or more image sensors (e.g., a front sensor or a rear sensor), lenses, an image signal processor (ISP), or a flash (e.g., an LED or a xenon lamp).

The power management module 295, for example, may manage power of the electronic device 201. According to an embodiment, the power management module 295 may include a power management integrated circuit (PMIC), a charger integrated circuit (IC), or a battery or fuel gauge. The PMIC may be implemented by a wired charging type and/or a wireless charging type. The wireless charging type may encompass, for example, a magnetic resonance type, a magnetic induction type, or an electromagnetic wave type, and additional circuits for wireless charging, such as coil loops, resonance circuits, or rectifiers, may be further included. The battery gauge may measure, for example, the remaining power of the battery 296, a charging voltage, current, or temperature. The battery 296 may include, for example, a rechargeable battery and/or a solar battery.

The indicator 297 may display a specific state (e.g., a booting state, a message state, or a charging state) of the whole or a part (e.g., the processor 210) of the electronic device 201. The motor 298 may convert an electric signal to a mechanical vibration, and may provide a vibration or a haptic effect. Although it is not shown in the drawing, the electronic device 201 may include a processing device (e.g., a GPU) for supporting mobile TV. The processing device for supporting mobile TV may process media data according to standards, such as, for example, digital multimedia broadcasting (DMB), digital video broadcasting (DVB), or mediaFlo™.

The respective elements described in the present specification may be comprised of one or more components, and the names of the elements may vary depending on the type of electronic device. According to various embodiments, the electronic device may be configured by including at least one of the elements described in the present specification, and may exclude some of the elements, or may further include other elements. In addition, some of the elements of the electronic device according to various embodiments may be combined to constitute a single entity, thereby performing the same function as that of the elements before being combined.

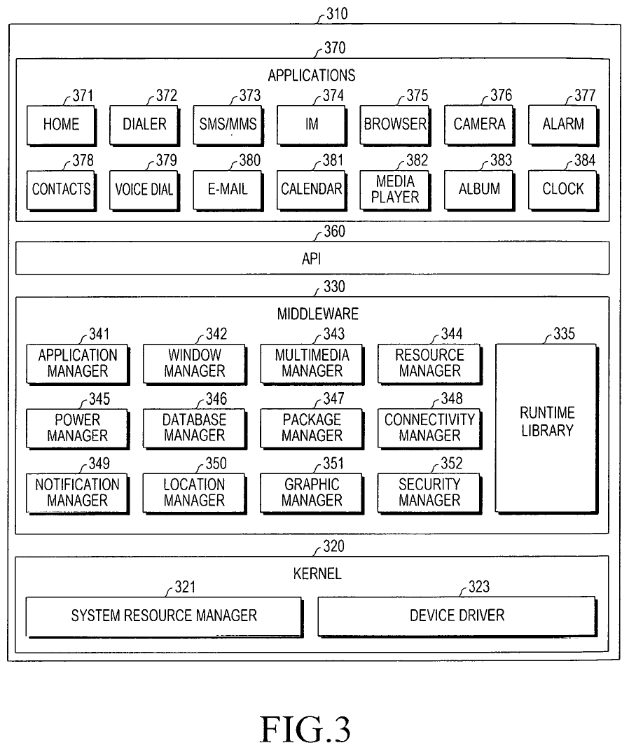

FIG. 3 is a block diagram of a program module, according to various embodiments. According to an embodiment, the program module 310 (e.g., the programs 140) may include an operating system (OS) for controlling resources related to the electronic device (e.g., the electronic device 101) and/or various applications (e.g., the application programs 147) that are operated under the operating system. For example, the operating system may be Android, iOS, Windows, Symbian, Tizen, Bada, or the like.

The program module 310 may include a kernel 320, middleware 330, an application programming interface (API) 360, and/or applications 370. At least some of the program module 310 may be preloaded in the electronic device, or may be downloaded from external electronic devices (e.g., the electronic devices 102 and 104 or the server 106).

The kernel 320 (e.g., the kernel 141), for example, may include a system resource manager 321 and/or a device driver 323. The system resource manager 321 may perform control, allocation, or collection of the system resources. According to an embodiment, the system resource manager 321 may include a process management unit, a memory management unit, or a file system management unit. The device driver 323 may include, for example, a display driver, a camera driver, a Bluetooth driver, a shared memory driver, a USB driver, a keypad driver, a Wi-Fi driver, an audio driver, or an inter-process communication (IPC) driver.

The middleware 330, for example, may provide functions required in common for the applications 370, or may provide the applications 370 with various functions through the API 360 in order to allow the applications 370 to effectively use the limited system resources in the electronic device. According to an embodiment, the middleware 330 (e.g., the middleware 143) may include at least one of a runtime library 335, an application manager 341, a window manager 342, a multimedia manager 343, a resource manager 344, a power manager 345, a database manager 346, a package manager 347, a connectivity manager 348, a notification manager 349, a location manager 350, a graphic manager 351, or a security manager 352.

The runtime library 335, for example, may include a library module that a compiler uses in order to add new functions through programming languages while the applications 370 are executed. The runtime library 335 may perform the input/output management, the memory management, or a function of an arithmetic calculation.

The application manager 341, for example, may manage a life cycle of at least one of the applications 370. The window manager 342 may manage a GUI resource used in the screen. The multimedia manager 343 may identify formats for reproducing various media files, and may perform encoding or decoding of media files using a codec corresponding to each format. The resource manager 344 may manage resources, such as a source code, a memory, or a storage space of at least one of the applications 370.

The power manager 345, for example, may manage a battery or power by operating in association with a basic input/output system (BIOS), and may provide power information necessary for the operation of the electronic device. The database manager 346 may create, retrieve, or change a database that is to be used in at least one of the applications 370. The package manager 347 may manage the installation or update of the applications that are distributed in the form of a package file.

The connectivity manager 348, for example, may manage a wireless connection, such as Wi-Fi or Bluetooth. The notification manager 349 may display or notify of events, such as received messages, appointments, or proximity notifications, to the user without disturbance. The location manager 350 may manage location information of the electronic device. The graphic manager 351 may manage graphic effects to be provided to the user or user interfaces related thereto. The security manager 352 may provide a general security function required for system security or user authentication. According to an embodiment, in the case of the electronic device (e.g., the electronic device 101) adopting a phone call function, the middleware 330 may further include a telephony manager for managing the functions of a voice call or a video call of the electronic device.

The middleware 330 may include a middleware module through a combination of various functions of the above-described elements. The middleware 330 may provide a module that is specialized according to the type of operating system in order to provide differentiated functions. In addition, the middleware 330 may dynamically exclude some of the typical elements or add new elements.

The API 360 (e.g., the API 145), for example, may be a set of API programming functions, and may be provided as a different configuration according to an operating system. For example, one set of APIs may be provided to each platform in the case of Android or iOS, and two or more sets of APIs may be provided to each platform in the case of Tizen.

The applications 370 (e.g., the application programs 147), for example, may include one or more applications that execute functions of home 371, a dialer 372, SMS/MMS 373, instant messages (IM) 374, a browser 375, a camera 376, an alarm 377, contacts 378, a voice dial 379, e-mail 380, a calendar 381, a media player 382, an album 383, a clock 384, healthcare (e.g., measuring the amount of exercise or blood glucose), providing environment information (e.g., providing atmospheric pressure, humidity, or temperature information), and the like.

According to an embodiment, the applications 370 may include an application (hereinafter, referred to as “information-exchange application” for the convenience of explanation) that supports the exchange of information between the electronic device (e.g., the electronic device 101) and the external electronic device (e.g., the electronic device 102 or 104). The information-exchange application, for example, may include a notification relay application for relaying specific information to the external electronic devices, or may include a device management application for managing the external electronic devices.

For example, the notification relay application may include a function of transferring notification information generated in other applications (e.g., the SMS/MMS application, the e-mail application, the healthcare application, or the environment information application) of the electronic device to the external electronic devices (e.g., the electronic devices 102 and 104). In addition, the notification relay application, for example, may receive notification information from the external electronic device to then provide the same to the user.

The device management application, for example, may manage (e.g., install, delete, or update): one or more functions {e.g., turning on and off the external electronic device (or some components) or adjusting the brightness (or resolution) of a display} of the external electronic device (e.g., the electronic device 102 or 104) that communicates with the electronic device; applications executed in the external electronic device; or services (e.g., a phone call service or a messaging service) provided by the external electronic device.

According to an embodiment, the applications 370 may include applications that are designated according to the attributes (e.g., the healthcare application of a mobile medical device) of the external electronic device (e.g., the electronic device 102 or 104). According to an embodiment, the applications 370 may include applications that are received from the external electronic device (e.g., the server 106 or the electronic device 102 or 104). According to an embodiment, the applications 370 may include preloaded applications or third party applications that may be downloaded from a server. The names of the elements of the program module 310, according to the illustrated embodiment, may vary depending on the type of operating system.

According to an embodiment, the applications 370 may include an application for determining the skin type of a user and providing at least one piece of related content according to the determined skin type.

According to various embodiments, at least some of the program module 310 may be implemented by software, firmware, hardware, or a combination of two or more thereof At least some of the program module 310, for example, may be implemented (e.g., executed) by the processor (e.g., the processor 120). At least some of the program module 310, for example, may include modules, programs, routines, sets of instructions, or processors for executing one or more functions.

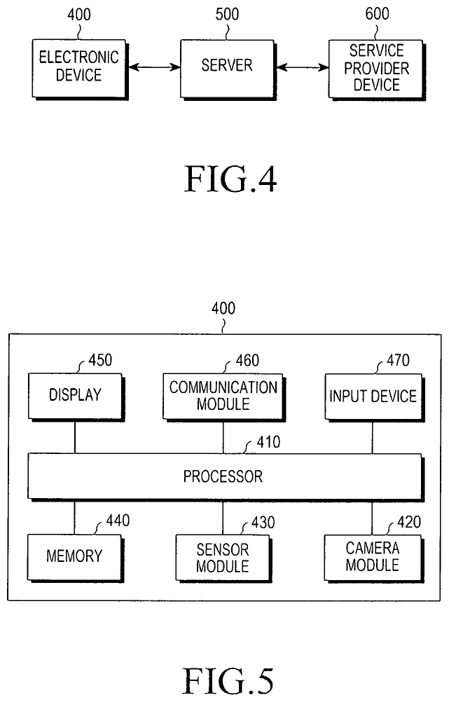

FIG. 4 illustrates a configuration diagram of a system for providing content depending on a skin type according to various embodiments:

The system may include an electronic device 400, a server 500, and a service provider device 600.

The electronic device 400 may obtain skin diagnosis information indicating the skin condition of a user, and, based on the obtained skin diagnosis information, may determine the skin type of the user. The user may be a content receiver who is provided with content (e.g., an image, a video, a webpage, internet search information, or the like, which includes product information, tips, community information, and the like in relation to the skin type) corresponding to his/her skin type using the electronic device 400.

According to an embodiment, the electronic device 400 may provide a user interface for receiving, from a user, replies to queries predetermined for skin diagnosis, and may analyze a user input (e.g., the replies to the queries) received through the provided user interface, thereby determining the skin type according to the analysis result. For example, the electronic device 400 may provide a user interface including queries for the skin condition of the user according to skin analysis items, such as wrinkles, pigmentation, trouble, and the like, and, when a user input on the queries is received through the user interface, may analyze the received user input, thereby determining the skin type according to the analysis result.

According to an embodiment, the electronic device 400 may include a camera module, and may photograph an image using the camera module. Then, the electronic device 400 may extract a face area from the photographed image, and may analyze the skin of at least a portion of the extracted face area, thereby determining the skin type according to the analysis result. For example, the electronic device 400 may analyze the skin corresponding to portions on the periphery of the eyes, nose, and mouth in the extracted face area according to skin analysis items, such as wrinkles, pigmentation, trouble, and the like, and may determine the skin type according to the analysis result.

According to an embodiment, the electronic device 400 may provide a user interface for receiving a user input regarding the skin condition from the user, may analyze the user input received through the provided user interface to thus generate first skin analysis information (e.g., a first skin characteristic value), and may analyze the skin using a camera module or a sensor module to thus generate second skin analysis information (e.g., a second skin characteristic value), thereby determining the skin type, based on the generated first and second skin analysis information.

According to various embodiments, the electronic device 400 may store content in a memory (e.g., the memory 130 or 230) according to a plurality of skin types, and may retrieve the content corresponding to the determined skin type from the memory, thereby providing the retrieved content. For example, if the determined skin type is a first skin type, the electronic device 400 may retrieve the content corresponding to the first skin type, and may display information on the first skin type and the retrieved content on the display.

According to various embodiments, the electronic device 400 may transmit a request for content related to the determined skin type to the server 500 and, when the content corresponding to the determined skin type is received from the server 500, may display the received content on the display. The content may be an image, a video, a webpage, internet search information, or the like, which includes product information, tips, community information, and the like in relation to the skin type.

The server 500 may store the content so as to correspond to a plurality of skin types. According to an embodiment, when the content is selected so as to correspond to a specific skin type by the service provider device 600, the server 500 may match the specific skin type with the selected content, and may then store the same. According to an embodiment, the server 500 may provide a content uploading and downloading function, and, when content is uploaded to correspond to a specific skin type from the service provider device 600, may store the uploaded content.

According to an embodiment, when a request for content related to the skin type is received from the electronic device 400, the server 500 may retrieve the requested content (the content related to the skin type) from stored content, and may transmit the retrieved content to the electronic device 400.

According to various embodiments, when a request for content related to the skin type is received, the server 500 may search for at least one keyword corresponding to the skin type through the Internet, and may transmit the search result (Internet search result) to the electronic device 400. For example, the server 500 may generate search information (e.g., a search keyword, a search formula, or the like) corresponding to the skin type and used for an Internet search, and, based on the generated search information, may search for the content related to the search information (e.g., Internet search results or Internet search information) through the Internet, thereby providing the searched content to the electronic device 400.

The service provider device 600 may select each piece of content related to at least one skin type. According to various embodiments, the service provider device 600 may match respective pieces of content with one or more skin types, and may transmit the same to the server 500. According to an embodiment, the service provider device 600 may upload the content corresponding to at least one skin type to the server 500.

FIG. 5 shows a configuration diagram of an electronic device according to various embodiments.

Referring to FIG. 5, the electronic device 400, for example, may include a processor 410, a camera module 420, a sensor module 430, a memory 440, a display 450, a communication module 460, and an input device 470.

The processor 410 may control the overall operation of the camera module 420, the sensor module 430, the memory 440, the display 450, the communication module 460, and the input device 470. The processor 410 may determine the skin type of a user, and may provide the content corresponding to the determined skin type.

According to an embodiment, the processor 410 may provide a user interface to receive a user input related to the skin condition from a user, may generate first skin analysis information by analyzing a user input received through the provided user interface, and, based on the generated first skin analysis information, may determine the skin type.

For example, the processor 410 may display, on the display 450, a user interface including an area for displaying queries to determine the skin condition of a user and an area for inputting replies to the queries.

When a user input on the queries is received from the input device 470 through the user interface, the processor 410 may analyze the received user input to obtain first skin analysis information, and, based on the obtained first skin analysis information, may determine the skin type.

The first skin analysis information may include a skin analysis value obtained by analyzing a user input according to at least one skin analysis item. For example, with regard to a query about whether or not the oil content of the skin of a user is equal to or more than a first threshold value, when a user input indicating that the oil content of the skin of a user is equal to or more than the first threshold value is received, an analysis value for oil may be “oil: equal to or more than a first threshold value”.

The processor 410 may store, in the memory 440, rating configuration information on respective skin analysis items in which ratings (e.g., 1/2/3, High/Medium/Low, Top/Middle/Bottom, or the like) are set so as to correspond to a plurality of threshold ranges (e.g., equal to or more than/more than a first threshold value, equal to or less than/less than a second threshold value, a first threshold range, a second threshold range, or the like) for respective skin analysis items. The rating configuration information on respective skin analysis items may be configured as a table. For example, the rating configuration information on respective skin analysis items may be configured as Table 1 and Table 2 below.

TABLE 1

| | High | Medium | Low |
|---|---|---|---|
| | (equal to or more | (equal to or more | (equal to or more |
| | than~less than) | than~less than) | than~less than) |
| Oil (%) | 70~100 | 30~70 | 0~30 |
| Moisture (%) | 70~100 | 30~70 | 0~30 |

TABLE 2

| | High | Low |
|---|---|---|
| | (equal to or more | (equal to or more |
| | than~less than) | than~less than) |
| Wrinkles (%) | 60~100 | 0~60 |
| Pigmentation (%) | 60~100 | 0~60 |
| Trouble (%) | 60~100 | 0~60 |

Accordingly, the processor 410, based on the rating configuration information on respective skin analysis items, may identify ratings for respective skin analysis items corresponding to the first skin analysis information, and may obtain skin rating information including the identified ratings for respective skin analysis items. For example, the skin rating information may include “Oil: High, Moisture: Medium, Wrinkles: Low, Pigmentation: Low, and Trouble: High”.

The processor 410 may determine the skin type corresponding to the obtained skin rating information, and may display information on the determined skin type and the content corresponding to the determined skin type on the display 450. For example, if the determined skin type is a first skin type, the first skin type may be expressed as a combination of letters and numbers (e.g., “C9”) in the form of a tag.

According to an embodiment, the processor 410 may analyze the skin of a user using at least one of the camera module 420 and the sensor module 430, and may determine the skin type according to the analysis result.

The processor 410 may analyze the skin corresponding to portions on the periphery of the eyes, nose, and mouth of the user according to at least one skin analysis item using the camera module 420 and the sensor module 430 to thus obtain second skin analysis information, and, based on the obtained second skin analysis information, may determine the skin type.

The second skin analysis information may include skin analysis values obtained by analyzing the skin of a user according to at least one skin analysis item using the camera module 420 and the sensor module 430. For example, if the oil content of the skin of a user is 80%, the skin analysis value for the oil item may be “Oil: 80%”.

The processor 410, based on the rating configuration information on respective skin analysis items, may identify the ratings for respective skin analysis items corresponding to the second skin analysis information, and may obtain skin rating information including the identified ratings for respective skin analysis items.

For example, in case where the ratings corresponding to a plurality of threshold ranges for the oil or moisture item are “High: equal to or more than 70% and less than 100%, Medium: equal to or more than 30% and less than 70%, and Low: equal to or more than 0% and less than 30%” in the rating configuration information on respective skin analysis items, if it is analyzed that the skin corresponding to a portion on the periphery of the eyes contains 70% oil and 30% moisture, the process 410 may identify a rating (e.g., High) corresponding to the oil item and a rating (e.g., Medium) corresponding to the moisture item, and may obtain skin rating information including the identified ratings for the oil and moisture items (e.g., oil: High and moisture: Medium).

The processor 410 may determine the skin type corresponding to the obtained skin rating information, and may display, on the display 450, information on the determined skin type and the content corresponding to the skin type.

According to an embodiment, the processor 410 may obtain first skin analysis information through the user interface, may obtain second skin analysis information of the user using at least one of the camera module 420 and the sensor module 430, and, based on the obtained first skin analysis information and second skin analysis information, may determine the skin type.

For example, the processor 410 may receive a user input related to the oil, moisture, wrinkles, pigmentation, trouble, and the like of the user through a user interface, may analyze the received user input, thereby obtaining first skin analysis information, and may analyze the skin of a user according to the skin analysis items, such as oil, moisture, wrinkles, pigmentation, trouble, and like of the user, using at least one of the camera module 420 and the sensor module 430, thereby obtaining second skin analysis information.

More specifically, the processor 410 may display, on the display 450, a user interface that includes an area for displaying queries for determining the skin type of a user and an area for inputting replies to the queries. When a user input on the queries is received from the input device 470 through the user interface, the processor 410 may analyze the input replies, thereby obtaining first skin analysis information.

The processor 410 may analyze the skin of a user according to the first skin analysis items, such as oil and moisture, and according to the second skin analysis items, such as wrinkles, pigmentation, trouble, and the like, using at least one of the camera module 420 and the sensor module 430, thereby obtaining second skin analysis information.

The processor 410, based on rating configuration information on respective skin analysis items, may identify one or more ratings for respective skin analysis items corresponding to the first skin analysis information and the second skin analysis information, and may obtain skin rating information including the one or more identified ratings for respective skin analysis items. For example, the skin rating information may include “Oil: High, Moisture: Medium, Wrinkle: Low, Pigmentation: Low, and Trouble: High”.

The processor 410 may determine the skin type corresponding to the obtained skin rating information, and may display information on the determined skin type and the content corresponding to the determined skin type on the display 450.

According to various embodiments, the processor 410 may obtain first skin analysis information through a user interface, may obtain second skin analysis information of the user using at least one of the camera module 420 and the sensor module 430, may obtain context information including at least one of user's environment information and health information, and, based on the first skin analysis information, the second skin analysis information, and the context information, may determine the skin type.

For example, the processor 410 may obtain current temperature information and humidity information using a temperature sensor or a humidity sensor, and may obtain environment information, such as weather, a UV index, fine dust concentration, and the like, from an environmental information provider, such as the National Weather Service, providing information on the weather, a UV index, fine dust concentration, and the like.

The processor 410 may analyze the obtained environment information according to environment analysis items, such as temperature, humidity, a UV index, fine dust concentration, and the like, thereby obtaining environment analysis information.

The processor 410 may store, in the memory 440, rating configuration information on respective environment analysis items in which ratings (e.g., High, Medium, and Low, or High and Low) are set to correspond to a plurality of threshold ranges (e.g., equal to or more than/more than a first threshold value, equal to or less than/less than a second threshold value, or the like) for respective environment analysis items. The rating configuration information for respective environment analysis items may be configured as a table. For example, the rating configuration information for respective environment analysis items may be configured as Table 3 below.

TABLE 3

| | High | Low |
|---|---|---|
| UV index (step) | 5 or more | less than 5 |
| Temperature (° C.) | 27 or more | less than 27 |
| Humidity (%) | 50 or more | less than 50 |
| Fine dust concentration | 60 or more | equal to or more than 0 and |
| (PM) | | less than 60 |

Accordingly, the processor 410, based on the rating configuration information for respective environment analysis items, may identify one or more ratings for respective environment analysis items corresponding to the environment analysis information, and may obtain environment rating information including the one or more identified ratings for respective environment analysis items.

For example, in the case where the ratings corresponding to a plurality of threshold ranges for a UV index item are “High: equal to or more than 5.0, and Low: less than 5.0” in the rating configuration table for respective environment analysis items, if the current UV index is “7.5”, the processor 410 may identify the rating (e.g., High) corresponding to the current UV index.

For example, in the case where the ratings corresponding to a plurality of threshold ranges for a temperature item are “High: equal to or more than 27 degrees C., and Low: less than 27 degrees C.” in the rating configuration information on respective environment analysis items, if the current temperature is “30 degrees C”, the processor 410 may identify the rating (e.g., High) corresponding to the current temperature.

For example, in the case where the ratings corresponding to a plurality of threshold ranges for a humidity item are “High: equal to or more than 50%, and Low: less than 50%” in the rating configuration information on respective environment analysis items, if the current humidity is “40%”, the processor 410 may identify the rating (e.g., Low) corresponding to the current humidity.

For example, in case where the ratings corresponding to a plurality of threshold ranges for a fine dust concentration item are “High: equal to or more than 60 and Low: equal to or more than 0 and less than 60” in the rating configuration information on respective environment analysis items, if the current fine dust concentration is “25”, the processor 410 may identify the rating (e.g., Low) corresponding to the current fine dust concentration.

Accordingly, the environment rating information may include one or more identified ratings for respective environment analysis items (e.g., “UV index: High, Temperature: High, Humidity: Low, and Fine dust concentration: Low”).

The processor 410 may determine the skin type corresponding to the skin rating information and environment rating information including “Oil: High, Moisture: Medium, Wrinkle: Low, Pigmentation: Low, Trouble: High, UV index: High, Temperature: High, Humidity: Low, and Fine dust concentration: Low”, and may display information on the determined skin type and the content corresponding to the determined skin type on the display 450. If the determined skin type is a second skin type, the second skin type may be expressed as a combination of letters and numbers such as “C94” in the form of a tag.

For example, the skin type may be expressed as a combination of letters or numbers corresponding to the skin rating information and the environment rating information with reference to Tables 4, 5, and 6 below. Table 4 represents a first skin type table including character information on respective ratings corresponding to the oil and moisture items, Table 5 represents a second skin type table including numeric information on respective ratings corresponding to the wrinkle, pigment, and trouble items, and Table 6 represents a third skin type table including numeric information on respective ratings corresponding to the UV index, temperature, humidity, and fine dust concentration items.

TABLE 4

| | Oil | Moisture |
|---|---|---|
| A | High | High |
| B | High | Medium |
| C | High | Low |
| D | Medium | High |
| E | Medium | Medium |
| F | Medium | Low |
| G | Low | High |
| H | Low | Medium |
| I | Low | Low |

TABLE 5

| | Wrinkles | Pigmentation | Trouble |
|---|---|---|---|
| 1 | High | High | High |
| 2 | High | High | Low |
| 3 | High | Low | High |
| 4 | High | Low | Low |
| 5 | Low | High | High |
| 6 | Low | High | Low |
| 7 | Low | Low | High |
| 8 | Low | Low | Low |

TABLE 6

| | | | | Fine dust |
|---|---|---|---|---|
| | UV index | Temperature | Humidity | concentration |
| 1 | High | High | High | High |
| 2 | High | High | High | Low |
| 3 | High | High | Low | High |
| 4 | High | High | Low | Low |
| 5 | High | Low | High | High |
| 6 | High | Low | High | Low |
| 7 | High | Low | Low | High |
| 8 | High | Low | Low | Low |
| 9 | Low | High | High | High |
| 10 | Low | High | High | Low |
| 11 | Low | High | Low | High |
| 12 | Low | High | Low | Low |
| 13 | Low | Low | High | High |
| 14 | Low | Low | High | Low |
| 15 | Low | Low | Low | High |
| 16 | Low | Low | Low | Low |

Based on the above Tables 4, 5, and 6, the skin type corresponding to “Oil: High, Moisture: Medium, Wrinkle: Low, Pigmentation: Low, Trouble: High, UV index: High, Temperature: High, Humidity: Low, and Fine dust concentration: Low” may be “B74”.

According to various embodiments, the processor 410 may store content corresponding to a plurality of skin types in the memory 440, and may retrieve the content corresponding to the determined skin type, thereby displaying the retrieved content on the display 450.

According to various embodiments, the processor 410 may transmit, to the server 500, a request for content related to the determined skin type, and, when the content corresponding to the determined skin type is received from the server 500, may display the received content on the display 450. For example, the content may include a text, an image, a document, a video, a webpage, and the like, in relation to the determined skin type.

The camera module 420 may photograph a still image and a moving image, and may output the photographed still image or moving image.

The sensor module 430 may include a sensor for measuring oil and moisture contained in the skin of a user, a sensor for measuring temperature or humidity, a UV sensor, and the like, and may output results measured using the sensors. For example, the oil/moisture sensor may measure the level of oil or moisture included in the skin of a user, and may output the measured result. Accordingly, the processor 410 may analyze the results measured by the oil/moisture sensor and the results measured by the temperature/humidity sensor and the UV sensor according to at least one skin analysis item.

The memory 440 may store commands or data related to an operation of providing content according to the skin type of a user in the electronic device 400. For example, the memory 440 may store content corresponding to a plurality of skin types.

The display 450 may display a user interface for receiving a user input according to the skin condition of a user. The display 450 may display the content corresponding to the determined skin type.

The communication module 460 may perform communication with the server 500. The communication module 460 may transmit a request for content related to the determined skin type to the server 500, and may receive content from the server 500.

The input device 470 may receive a user input according to the skin condition of the user.

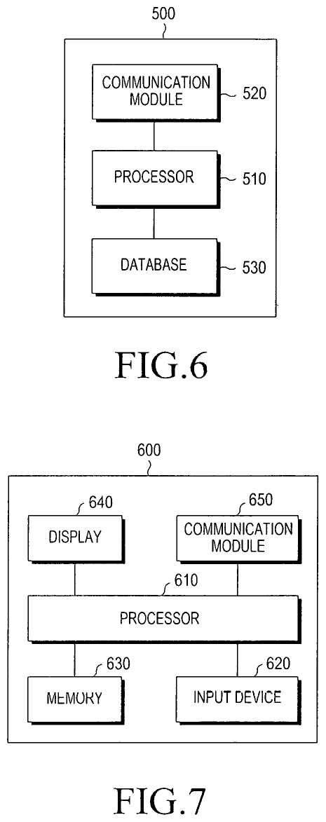

FIG. 6 shows a configuration diagram of a server according to various embodiments.

Referring to FIG. 6, a server 500 may include a processor 510, a communication module 520, and a database 530.

When a request for matching a specific skin type with content is received from a service provider device 600 via the communication module 520, the processor 510 may match a specific skin type with content according to the request of the service provider device 600, and may store the same in the database 530.

According to various embodiments, the processor 510 may provide an upload or download function for content, and when the content corresponding to a specific skin type is uploaded by the service provider device 600, the processor 510 may match the specific skin type with the content to then store the same in the database 530.

When a request for the content corresponding to a specific skin type is received from the electronic device 400 through the. communication module 520, the processor 510 may transmit the content corresponding to the specific skin type to the electronic device 400.

According to various embodiments, when a request for downloading the content corresponding to .a specific skin type is received from the electronic device 400, the processor 510 may transmit the content corresponding to the specific skin type to the electronic device 400.

The communication module 520 may receive a request for the content corresponding to a specific skin type from the electronic device 400, and may transfer the content corresponding to the specific skin type to the electronic device 400.

The database 530 may store content corresponding to a plurality of skin types. .

FIG. 7 illustrates a configuration diagram of a service provider device according to various embodiments.

Referring to FIG. 7, the service provider device 600 may include a processor 610, an input device 620, a memory 630, a display 640, and a communication module 650.

When content related to a specific skin type is selected through the input device 620, the processor 610 may transmit a request for matching the specific skin type with the selected content to the server 500 through the communication module 650. The content may be text, images, documents, videos, webpages, Internet search information, or the like, which contain information on the products, tips, community information, and the like in relation to the specific skin type

For example, when an image for a first cosmetic product is selected to correspond to a first skin type (e.g., C47) through an input device 620, the processor 610 may transmit a request for matching the first skin type with the image for the first cosmetic product to the server 500 through the communication module 650.

According to various embodiments, the processor 610 may match the image of the first cosmetic product selected to correspond to the first skin type with the first skin type, and may transmit the image of the first cosmetic product, which has been matched with the first skin type, to the server 500 through the communication module 650.

According to various embodiments, the processor 610 may provide a user interface for selecting (or uploading) content related to a plurality of skin types. For example, the processor 610 may display, on the display 640, a user interface for selecting a specific skin type from among a plurality of skin types and for uploading content related to the selected skin type. When the content is selected through the displayed user interface, the processor 610 may transmit a request for matching a specific skin type with the selected content to the server 500, and may upload the selected content to the server 500.

According to various embodiments, the processor 610 may provide a user interface for matching a plurality of skin types with content related to the skin types and for uploading the matched content to the server 500. For example, the processor 610 may display the user interface on the display 640. When a specific skin type and content related to the specific skin type are selected through the displayed user interface, the processor 610 may match the specific skin type with the selected content, and may transmit the content matched with the specific skin type to the server 500 through the communication module 650.

The input device 620 may receive an input for selecting content related to a specific skin type, or may receive a request for matching a specific skin type with the selected content.

The memory 630 may store commands or data related to an operation of matching the skin type of a user with the content related to the corresponding skin type in the service provider device 600. For example, the memory 630 may store content related to a specific skin type.

The display 640 may display a user interface for selecting content related to a plurality of skin types.

The communication module 650 may transmit a request for matching a specific skin type with content to the server 500, or may transmit the content matched with a specific skin type to the server 500.

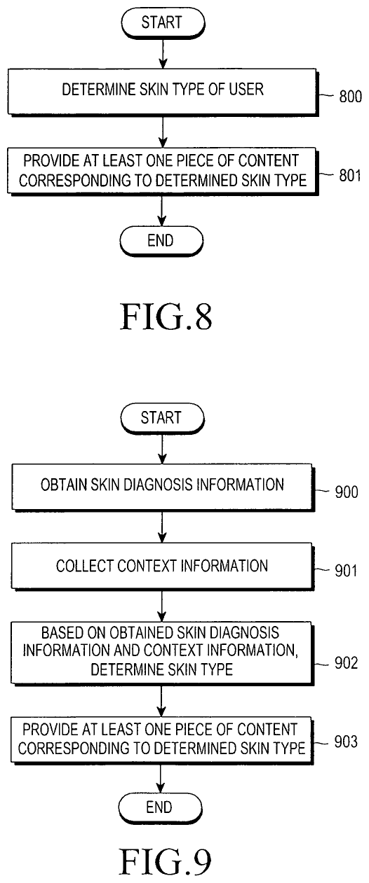

FIG. 8 illustrates a flowchart showing a process for providing content related to a skin type in an electronic device according to various embodiments. According to various embodiments, operations 800 and 801 may be performed by the electronic device 101, 104, 201 or 400, the server 106 or 500, the processor 120, 210, 410, 510, or 610, the service provider device 600, and the program module 310.

Referring to FIG. 8, in operation 800, the electronic device 400 (e.g., the processor 410) may determine the skin type of a user.

According to an embodiment, the electronic device 400 (e.g., the processor 410) may obtain skin diagnosis information indicating the skin condition of a user, and, based on the obtained skin diagnosis information, may determine the skin type of a user.

For example, the electronic device 400 (e.g., the processor 410) may receive, from the user, replies to queries predetermined for skin diagnosis, and may analyze the received user input according to at least one skin analysis item (e.g., oil, moisture, wrinkles, pigmentation, trouble, or the like), thereby determining the skin type according to the analysis result.

For example, the electronic device 400 (e.g., the processor 410) may analyze the skin of a user according to at least one skin analysis item using the camera module 420 or the sensor module 430, and may determine the skin type according to the analysis result.

For example, electronic device 400 (e.g., the processor 410) may receive a user input on the skin condition of a user, may analyze the received user input according to at least one skin analysis item, may analyze the skin of a user according to at least one skin analysis item using the camera sensor 420 or the sensor module 430, and may determine the skin type according to both analysis results above.

According to various embodiments, the electronic device 400 (e.g., the processor 410) may obtain skin diagnosis information representing the skin condition of a user, may collect context information indicating the environment state of a user, and may analyze the skin diagnosis information and the collected context information, thereby determining the skin type according to the analysis result. The context information may include at least one piece of environment information and health information of a user.

For example, the electronic device 400 (e.g., the processor 410) may collect environment information on at least one of a UV index, fine dust concentration, temperature, humidity, or the like, which indicates the environment state of a user, and may analyze the collected environment information. The electronic device 400 (e.g., the processor 410) may collect health information on at least one of the amount of calorie consumption, the amount of moisture intake, sleeping time, stress, and the like, which indicates the health condition of a user, and may analyze the collected health information.

As described above, based on the analyzed skin diagnosis information and the analyzed environment and health information, the electronic device 400 (e.g., the processor 410) may determine the skin type.

In operation 801, the electronic device 400 (e.g., the processor 410) may provide at least one piece of content corresponding to the determined skin type.

According to an embodiment, the electronic device 400 (e.g., the processor 410) may store, in the memory 440, content corresponding to a plurality of skin types, may retrieve the content corresponding to the determined skin type, and may display information on the determined skin type and the retrieved content on the display 450.

According to an embodiment, the electronic device 400 (e.g., the processor 410) may transmit a request for the content corresponding to the determined skin type to the server 500, and, when the content corresponding to the determined skin type is received from the server 500, may display information on the determined skin type and the received content on the display 450.

FIG. 9 illustrates a flowchart in which an electronic device, based on skin diagnosis information and context information, provides content related to a skin type according to various embodiments. According to various embodiments, operations 900 to 903 may be performed by the electronic device 101, 104, 201 or 400, the server 106 or 500, the processor 120, 210, 410, 510, or 610, the service provider device 600, and the program module 310.

Referring to FIG. 9, in operation 900, the electronic device 400 (e.g., the processor 410) may obtain skin diagnosis information.

According to an embodiment, the electronic device 400 (e.g., the processor 410) may provide a user interface for receiving, from a user, replies to queries predetermined for skin diagnosis, and may analyze a user input (e.g., replies to queries) received through the user interface, thereby obtaining first skin analysis information. For example, the electronic device 400 may obtain first skin analysis information by analyzing the user input according to at least one skin analysis item such as oil, moisture, wrinkles, pigmentation, trouble, or the like.

According to an embodiment, the electronic device 400 (e.g., the processor 410) may photograph an image using the camera module 420, and may analyze the skin of a face area of the photographed image according to at least one skin analysis item, or may analyze the skin of a user using the sensor module 430, thereby obtaining second skin analysis information. For example, the electronic device 400 (e.g., the processor 410) may analyze the skin of a user according to at least one skin analysis item, such as oil, moisture, wrinkles, pigmentation, trouble, or the like, thereby obtaining the second skin analysis information.

The electronic device 400 (e.g., the processor 410), based on rating configuration information on respective skin analysis items, may identify ratings for respective skin analysis items corresponding to the first skin analysis information and the second skin analysis information, and may obtain skin rating information including the identified ratings for respective skin analysis items.

For example, in the case where ratings according to a plurality of threshold ranges corresponding to oil and water items are “High: 70% or more and less than 100%, Medium: 30% or more and less than 70%, and Low: 0% or more and less than 30%” in the rating configuration information on respective skin analysis items, if the oil content is 90% and the moisture content is 30% in the first skin analysis information, the electronic device 400 (e.g., the processor 410) may identify the rating for (e.g., High) the oil item and the rating (e.g., Medium) of the moisture item.

In addition, in the case where ratings according to a plurality of threshold ranges corresponding to wrinkle, pigmentation, and trouble items arc “High: 60% or more and less than 100% and Low: 0% or more and less than 60%” in the rating configuration information on respective skin analysis items, if the skin analysis information has the wrinkle content of 20%, the pigmentation content of 50%, and the trouble content of 10%, the electronic device 400 may identify the rating (e.g., Low) for the wrinkles item, the rating (e.g., Low) of the pigmentation item, and the rating (e.g., Low) for the trouble item.

The electronic device 400 (e.g., the processor 410) may obtain skin rating information including the identified ratings (e.g., “Oil: High, Water: Medium, Wrinkles: Low, Pigmentation: Low, and Trouble: Low”).

In operation 901, the electronic device 400 (e.g., the processor 410) may collect context information.

According to an embodiment, the electronic device 400 (e.g., the processor 410) may obtain environment information on a UV index, fine dust concentration, temperature, humidity, and the like through an environment information provider, such as the National Weather Service, or a temperature/humidity sensor, and may analyze the obtained environment information according to at least one environment analysis item, thereby obtaining environment analysis information. The environment analysis information may include one or more environment analysis values for respective environment analysis items. For example, an environment analysis value for the temperature item may be the current temperature (e.g., 20 degrees C.).

The electronic device 400 (for example, the processor 410), based on rating configuration information on respective environment analysis items, may identify ratings for respective environment analysis items corresponding to the environment analysis information, and may obtain environment rating information including the identified ratings for respective environment analysis items.

For example, in the case where the ratings corresponding to a plurality of threshold ranges for a temperature item are “High: equal to or more than 27 degrees C. and Low: less than 27 degrees C.” in the rating configuration information on respective environment analysis items, if an analysis value for the temperature item is “30 degrees C.” in the environment analysis information, the electronic device 400 may identify the rating (e.g., High) corresponding to the analysis value of the temperature item (e.g., 30 degrees C.).

The electronic device 400 (e.g., the processor 410), based on the rating configuration information on respective environment analysis items, may identify the ratings corresponding to analysis values for a UV index item, a humidity item, and a fine dust concentration item, as well as the temperature item, and may obtain environment rating information including the identified ratings.

In operation 902, based on the obtained skin diagnosis information and the obtained context information, the electronic device 400 (e.g., the processor 410) may determine the skin type.

For example, if the skin rating information obtained based on the rating configuration information on respective skin analysis items and the environment rating information obtained based on the rating configuration information on respective environment analysis items “Oil: High, Moisture: Medium, Wrinkles: Low, Pigmentation: Low, Trouble: High, UV Index: High, Temperature: High, Humidity: Low, and Fine dust concentration: Low”, the electronic device 400 (e.g., the processor 410) may determine the skin type corresponding to “Oil: High, Moisture: Medium, Wrinkles: Low, Pigmentation: Low, Trouble: High, UV Index: High, Temperature: High, Humidity: Low, and Fine dust concentration: Low”.

In operation 903, the electronic device 400 (e.g., the processor 410) may provide at least one piece of the content corresponding to the determined skin type.

According to an embodiment, the electronic device 400 (e.g., the processor 410) may store content according to a plurality of skin types, may retrieve the content corresponding to the determined skin type, and display information on the determined skin type and the retrieved content on the display 450.

According to an embodiment, the electronic device 400 (e.g., the processor 410) may transmit a request for the content corresponding to the determined skin type to the server 500, and, when the content corresponding to the skin type is received from the server 500, may display information on the determined skin type and the received content on the display 450.

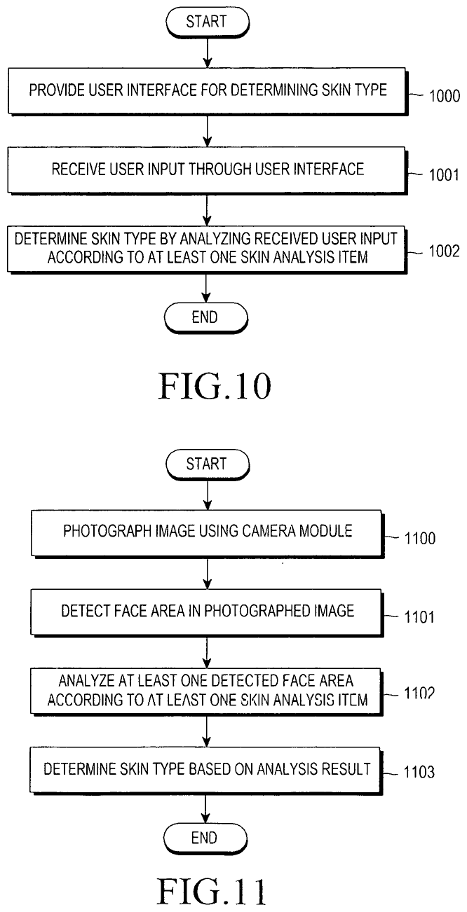

FIG. 10 illustrates a flowchart showing a process for determining a skin type using a user interface to receive, from a user, replies to queries predetermined for skin diagnosis according to various embodiments. According to various embodiments, operations 1000 to 1002 may be performed by the electronic device 101, 104, 201 or 400, the server 106 or 500, the processor 120, 210, 410, 510, or 610, the service provider device 600, and the program module 310.

Referring to FIG. 10, in operation 1000, the electronic device 400 (e.g., the processor 410) may provide a user interface for determining a skin type.

According to an embodiment, the electronic device 400 (e.g., the processor 410) may provide a user interface for receiving, from a user, replies to queries predetermined for skin diagnosis. The user interface may include an area for displaying queries for determining the skin condition of a user and an area for inputting replies to the queries.

In operation 1001, the electronic device 400 (e.g., the processor 410) may receive a user input through the user interface. For example, the electronic device 400 (e.g., the processor 410) may receive a user input indicating replies to queries through the user interface.

In operation 1002, the electronic device 400 (e.g., the processor 410) may analyze the received user input according to at least one skin analysis item, thereby determining the skin type.

According to an embodiment, the electronic device 400 (e.g., the processor 410) may analyze the received user input to thus obtain first skin analysis information, and, based on the obtained first skin analysis information, may determine the skin type.

For example, when a reply (e.g., selecting “YES” from among “YES and NO”) to a query for the skin condition of a user (e.g., “is the oil content of your skin equal to or more than a first threshold value?”) is received, the electronic device 400 (e.g., the processor 410) may obtain an analysis value corresponding to the oil item (e.g., oil: a first threshold value or more), and, based on rating configuration information on respective skin analysis items, may identify the rating (e.g., High) corresponding to the obtained analysis value. The electronic device 400 (e.g., the processor 410) may obtain analysis values corresponding to other skin analysis items, as well as the oil item, may identify ratings corresponding to the obtained analysis values, and may determine the skin type corresponding to skin rating information including the identified ratings.

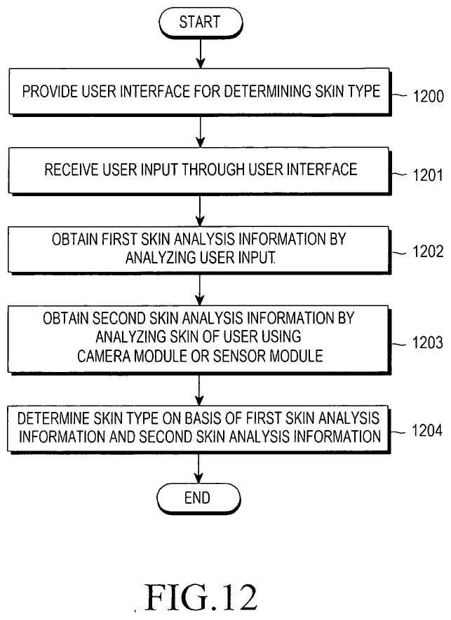

FIG. 11 illustrates a flowchart showing a process in which an electronic device determines a skin type using a camera module according to various embodiments. According to various embodiments, operations 1100 to 1103 may be performed by the electronic device 101, 104, 201 or 400, the server 106 or 500, the processor 120, 210, 410, 510, or 610, the service provider device 600, and the program module 310.

Referring to FIG. 11, in operation 1100, the electronic device 400 (e.g., the processor 410) may photograph an image using the camera module 420.

In operation 1101, the electronic device 400 (e.g., the processor 410) may detect at least one face area in the photographed image.

In operation 1102, the electronic device 400 (e.g., the processor 410) may analyze the detected face area according to at least one skin analysis item.

According to an embodiment, the electronic device may analyze the skin corresponding to at least a portion on the periphery of the eyes, nose, mouth, and the like in the detected face area according to at least one skin analysis item, such as wrinkles, pigmentation, trouble, or the like, thereby obtaining second skin analysis information.

More specifically, processor 410 (e.g., the processor 410) may analyze at least one of the thickness, size, number, area, or color of the skin analysis item(s), such as wrinkles, pigmentation, trouble, or the like, thereby obtaining the second skin analysis information including analysis values for the respective skin analysis items. The analysis values may include a value obtained by quantifying at least one of the thickness, size, number, area, and color of the wrinkles, pigmentation, trouble, or the like

In operation 1103, the electronic device 400 (e.g., the processor 410), based on the analysis result, may determine the skin type.

According to an embodiment, the electronic device 400 (e.g., the processor 410), based on rating configuration information on respective skin analysis items, may identify the ratings for respective skin analysis items corresponding to the second skin analysis information, and may obtain skin rating information including the ratings for respective skin analysis items.

If the obtained skin rating information is “Wrinkles: High, Pigmentation: high, and Trouble: Low”, the electronic device 400 (e.g., the processor 410) may identify the skin type corresponding to “Wrinkles: High, Pigmentation: High, and Trouble: Low”, and may identify text information or numeric information on each rating corresponding to the wrinkles, pigmentation, and trouble items, thereby determining the skin type by combining the identified text information or numeric information.

FIG. 12 illustrates a flowchart showing a process in which an electronic device determines the skin type using a user interface for determining the skin type of a user and a camera module or a sensor module according to various embodiments. According to various embodiments, operations 1200 to 1204 may be performed by the electronic device 101, 104, 201 or 400, the server 106 or 500, the processor 120, 210, 410, 510, or 610, the service provider device 600, and the program module 310.

Referring to FIG. 12, in operation 1200, the electronic device 400 (e.g., the processor 410) may provide a user interface for determining a skin type. According to an embodiment, the electronic device 400 (e.g., the processor 410) may provide a user interface for receiving, from a user, replies to queries predetermined for skin diagnosis.

In operation 1201, the electronic device 400 (e.g., the processor 410) may receive a user input through the user interface. For example, the electronic device 400 (e.g., the processor 410) may receive a user input that represents replies to one or more queries for respective skin analysis items through the user interface.

In operation 1202, the electronic device 400 (e.g., the processor 410) may analyze the user input, thereby obtaining first skin analysis information. For example, the electronic device 400 (e.g., the processor 410) may analyze replies to one or more queries for respective skin analysis items, thereby obtaining first skin analysis information including analysis one or more values for respective skin analysis items.

In operation 1203, the electronic device 400 (e.g., the processor 410) may analyze the skin of a user using the camera module 420 or the sensor module 430, thereby obtaining second skin analysis information.

According to an embodiment, the electronic device 400 (e.g., the processor 410) may photograph an image through the camera module 420, and may detect a face area in the photographed image. The electronic device 400 (e.g., the processor 410) may analyze the skin corresponding to the detected face area according to at least one skin analysis item, or may analyze the skin of a user using the sensor module 430 according to at least one skin analysis item, thereby obtaining second skin analysis information including one or more analysis values for respective skin analysis items.

In operation 1204, the electronic device 400 (e.g., the processor 410), based on the first skin analysis information and the second skin analysis information, may determine the skin type.

According to an embodiment, based on the rating configuration information on respective skin analysis items, the electronic device 400 (e.g., the processor 410) may determine the ratings corresponding to the first skin analysis information and the second skin analysis information. For example, electronic device 400 (e.g., the processor 410) may identify the ratings corresponding to the analysis values for respective skin analysis items including oil, moisture, wrinkles, pigmentation, trouble, or the like.

The electronic device 400 (e.g., the processor 410) may determine the skin type corresponding to the skin rating information including the identified ratings. For example, the electronic device 400 (e.g., the processor 410) may determine the skin type including a combination of text information or numeric information on the skin analysis items.

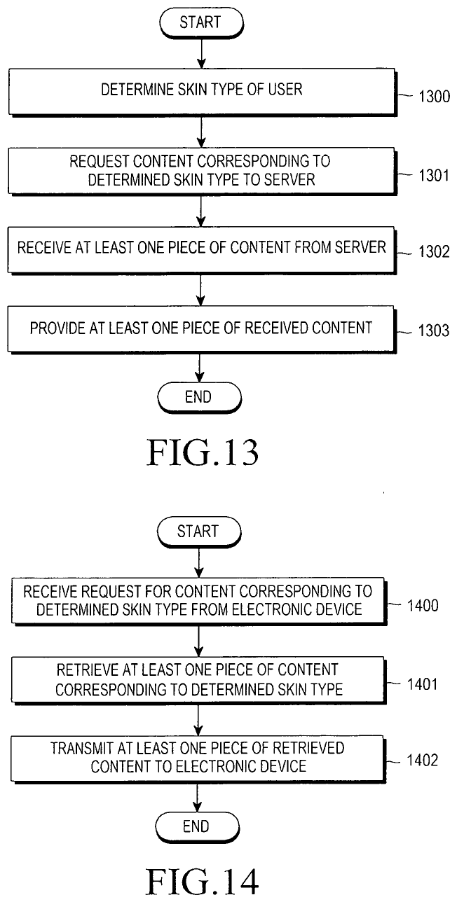

FIG. 13 illustrates a flowchart showing a process in which an electronic device receives content corresponding to a skin type from a server and provides the same according to various embodiments. According to various embodiments, operations 1300 to 1303 may be performed by the electronic device 101, 104, 201 or 400, the server 106 or 500, the processor 120, 210, 410, 510, or 610, the service provider device 600, and the program module 310.

Referring to FIG. 13, in operation 1300, the electronic device 400 (e.g., the processor 410) may determine the skin type of a user. According to an embodiment, the electronic device 400 (e.g., the processor 410) may provide a user interface for receiving, from a user, replies to queries predetermined for skin diagnosis. The electronic device 400 (e.g., the processor 410) may analyze a user input received through the provided user interface, or may analyze the skin of a user using the camera module 420 or the sensor module 430, thereby determining the skin type corresponding to the analysis result.

In operation 1301, the electronic device 400 (e.g., the processor 410) may make a request to the server 500 for the content corresponding to the determined skin type. For example, the electronic device 400 (e.g., the processor 410) may transmit, to the server 500, a request message that includes the determined skin type (e.g., C47) and requests the content corresponding to the skin type.

In operation 1302, the electronic device 400 (e.g., the processor 410) may receive content from the server 500. For example, the electronic device 400 (e.g., the processor 410) may receive a response message including the content corresponding to the determined skin type (e.g., a document, an image, a video, or a webpage corresponding to the determined skin type).

In operation 1303, the electronic device 400 (e.g., the processor 410) may provide at least one piece of the received content. For example, the electronic device 400 (e.g., the processor 410) may display, on the display 450, information on the determined skin type and the received content. The content may include, for example, documents, images, videos, or webpages for skin care tips of the skin type “C47”, skin care information proposed by a role model of the skin type “C47”, product information related to the skin type “C47”, such as cosmetic products, community information on users of the skin type “C47”, and the like, which are matched to correspond to the skin type “C47” by the service provider device 600.

FIG. 14 illustrates a flowchart showing a process in which a server provides the content corresponding to a skin type upon request by an electronic device according to various embodiments. According to various embodiments, operations 1400 to 1402 may be performed by the electronic device 101, 104, 201 or 400, the server 106 or 500, the processor 120, 210, 410, 510, or 610, the service provider device 600, and the program module 310.

Referring to FIG. FIG. 14, in operation 1400, the server 500 (e.g., the processor 510) may receive a request for the content corresponding to the determined skin type from the electronic device 400. For example, the server 500 (e.g., the processor 510) may receive, from the electronic device 400, a request message that includes the determined skin type and requests the content corresponding to the skin type

In operation 1401, the server 500 (e.g., the processor 510) may retrieve at least one piece of the content corresponding to the determined skin type. For example, the server 500 (e.g., the processor 510) may store content correspond to a plurality of skin types in the database 530, and may retrieve the content corresponding to the skin type received from the electronic device 400 from the stored content.

In operation 1402, the server 500 (e.g., the processor 510) may transmit at least one piece of the retrieved content to the electronic device 400. For example, the server 500 (e.g., the processor 510) may transmit, to the electronic device 400, a response message including the content that has been retrieved to correspond to the skin type and has been received from the electronic device 400 in response to the request message received from the electronic device 400.

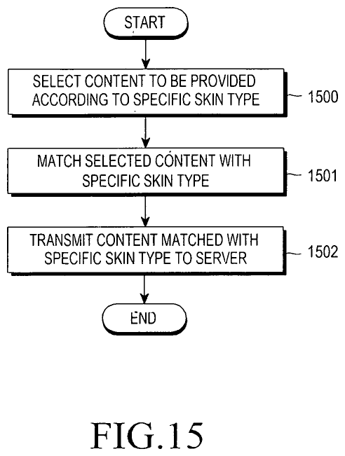

FIG. 15 illustrates a flowchart showing a process in which a service provider device matches at least one skin type with content according to various embodiments. According to various embodiments, operations 1500 to 1502 may be performed by the electronic device 101, 104, 201 or 400, the server 106 or 500, the processor 120, 210, 410, 510, or 610, the service provider device 600, and the program module 310.

Referring to FIG. 15, in operation 1500, the service provider device 600 (e.g., the processor 610) may select content to be provided according to a specific skin type.

According to an embodiment, the service provider device 600 (e.g., the processor 610) may select content related to at least one skin type.

For example, the service provider device 600 (e.g., the processor 610) may display, on the display 610, a user interface including a first area for selecting at least one skin analysis item and a second area for selecting a rating for at least one selected skin analysis item. The user interface may further include a third area for selecting at least one environment analysis item, and a fourth area for selecting a rating for at least one selected environment analysis item. The first area may include an area for selecting skin analysis items, such as oil, moisture, wrinkles, pigmentation, and trouble, and the second area may include an area for selecting ratings, such as High, Medium, and Low or High and Low, for the respective skin analysis items such as oil, wrinkles, pigmentation, trouble, and the like. In addition, the third area may include an area for selecting environment analysis items, such as a UV index, temperature, humidity, fine dust concentration, and the like, and the fourth area may include an area for selecting ratings, such as High, Medium, and Low or High and Low, for the respective environment analysis items such as a UV index, temperature, humidity, fine dust concentration, and the like.

When the selection for at least one skin analysis item is input through the first area of the user interface, and when the selection for a rating of at least one skin analysis item is input through the second area, the service provider device 600 (e.g., the processor 610) may display information on the skin type corresponding to the selected rating for at least one selected skin analysis item, and may activate and display a fifth area for selecting the content corresponding to the displayed skin type. The fifth area may include an area for selecting or uploading the content corresponding to the displayed skin type.

For example, if “Oil: High, Moisture: Medium, Wrinkles: Low, Pigmentation: Low, and Trouble: High” are selected through the user interface, the service provider device 600 (e.g., the processor 610) may display information on the skin type (e.g., C9) corresponding to selected “Oil: High, Moisture: Medium, Wrinkles: Low, Pigmentation: Low, and Trouble: High”.

According to various embodiments, when the selection for at least one environment analysis item is input through the third area, and when selection for a rating of at least one environment analysis item is input through the fourth area, in addition to the inputs through the first area and the second area, the service provider device 600 (e.g., the processor 610) may display information on the skin type corresponding to the rating for at least one skin analysis item and the rating for at least one environment analysis item, and may display a fifth area for selecting the content corresponding to the displayed skin type.

For example, if “Oil: High, Moisture: Medium, Wrinkles: Low, Pigmentation: Low, Trouble: High, UV index: High, Temperature: High, Humidity: Medium, and Fine dust concentration: Low” are selected through the user interface, the service provider device 600 may display information on the skin type (e.g., C94) corresponding to “Oil: High, Moisture: Medium, Wrinkles: Low, Pigmentation: Low, Trouble: High, UV index: High, Temperature: High, Humidity: Medium, and Fine dust concentration: Low”.

Accordingly, the user may select or upload the content (e.g., an image, a document, a video, a webpage link, or the like) corresponding to the skin type through the activated fifth area.

In operation 1501, the service provider device 600 (e.g., the processor 610) may match the selected content with the specific skin type. According to an embodiment, if selection for the content corresponding to the skin type is received through the fifth area, the service provider device 600 may match the displayed skin type with the selected content. For example, when a “first image” for the cosmetic product related to the skin type “C94” is selected through the fifth area, the service provider device 600 may match the “first image” with “C94” to then store the same in the memory 630.

In operation 1502, the service provider device 600 (e.g., the processor 610) may transmit the content matched with a specific skin type to the server 500. For example, the service provider device 600 may transmit the content (e.g., the first image) matched with the displayed skin type (e.g., “C94”) to the server 500.

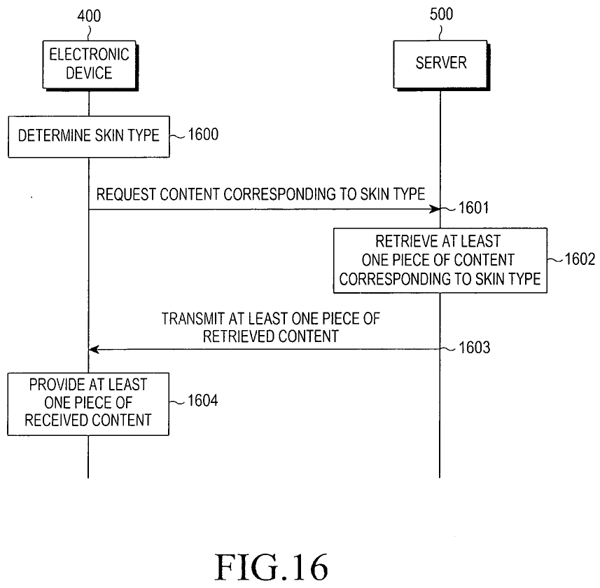

FIG. 16 illustrates a flowchart showing a process for providing content corresponding to a skin type between an electronic device and a server according to various embodiments. According to various embodiments, operations 1600 to 1604 may be performed by the electronic device 101, 104, 201 or 400, the server 106 or 500, the processor 120, 210, 410, 510, or 610, the service provider device 600, and the program module 310.

Referring to FIG. 16, in operation 1600, the electronic device 400 (e.g., the processor 410) may determine the skin type of a user. According to an embodiment, the electronic device 400 (e.g., the processor 410) may provide a user interface for receiving, from a user, replies to queries predetermined for skin diagnosis, may analyze a user input received through the provided user interface, or may analyze the skin of a user using the camera module 420 or the sensor module 430, thereby determining the skin type corresponding to the analysis result.

In operation 1601, the electronic device 400 (e.g., the processor 410) may make a request to the server 500 for the content corresponding to the skin type. For example, the electronic device 400 (e.g., the processor 410) may transmit, to the server 600, a request message that includes the determined skin type and requests the content corresponding to the skin type.

In operation 1602, the server 500 (e.g., the processor 510) may retrieve the content corresponding to the skin type. For example, the server 500 may store content corresponding to one or more skin types in the database, and may retrieve the content corresponding to the skin type received from the electronic device 400.

In operation 1603, the server 500 (e.g., the processor 510) may transmit the retrieved content to the electronic device 400. For example, the server 500 (e.g., the processor 510) may transmit a response message containing the retrieved content to the electronic device 400.

In operation 1604, the electronic device 400 (e.g., the processor 410) may provide the content received from the server 500. For example, the electronic device 400 (e.g., the processor 410) may display, on the display 450, information on the skin type and the content contained in the response message received from the server 500.

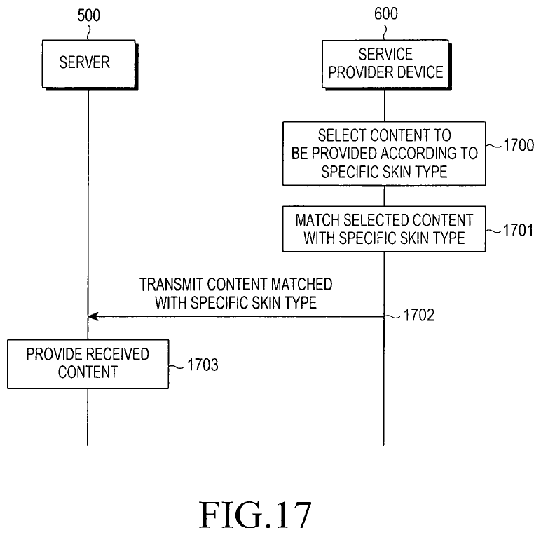

FIG. 17 illustrates a flowchart showing a process of matching the content corresponding to a skin type between a server and a service provider device according to various embodiments. According to various embodiments, operations 1700 to 1703 may be performed by the electronic device 101, 104, 201 or 400, the server 106 or 500, the processor 120, 210, 410, 510, or 610, the service provider device 600, and the program module 310.

Referring to FIG. 17, in operation 1700, the service provider device 600 (e.g., the processor 610) may select content to be provided according to a specific skin type. For example, the service provider device 600 (e.g., the processor 610) may provide a user interface for selecting content according to a specific skin type, and may select the content corresponding to the specific skin through the user interface. The user interface may include a first area for selecting at least one skin analysis item and a second area for selecting a rating for at least one selected skin analysis item. The user interface may further include a third area for selecting at least one environment analysis item and a fourth area for selecting a rating for at least one selected environment analysis item.

When at least one skin analysis item is selected, and when a rating for at least one selected skin analysis item is selected through the user interface, the service provider device 600 (e.g., the processor 610) may display information on the skin type corresponding to skin rating information including the selected rating for at least one skin analysis item, and may activate and display, in the user interface, a fifth area for selecting content corresponding to the displayed information on the skin type.

Accordingly, the user may select or upload the content (e.g., an image, a document, a video, a webpage link, or the like) corresponding to the skin type through the activated fifth area.

In operation 1701, the service provider device 600 (e.g., the processor 610) may match the selected content with the skin type. According to an embodiment, when the content corresponding to a specific type is selected through the fifth area, the service provider device 600 (e.g., the processor 610) may match the selected content with the skin type. For example, the service provider device 600 (e.g., the processor 610) may match the selected content with skin rating information corresponding to the skin type.

In operation 1702, the service provider device 600 (e.g., the processor 610) may transmit the content matched with the specific skin type to the server 500. For example, the service provider device 600 (e.g., the processor 610) may include skin rating information corresponding to the specific skin type in the header of the content, thereby transmitting the same to the server 500.

In operation 1703, the server 500 (e.g., the processor 510) may provide the content received from the service provider device 600. According to an embodiment, the server 500 (e.g., the processor 510) may transmit the content matched with the specific skin type to the electronic device 400 upon request by the electronic device 400. For example, the server 500 (e.g., the processor 510) may identify the skin type corresponding to the skin rating information included in the header of the content, and may store the content corresponding to the identified skin type. When a request for the content corresponding to the specific skin type is received from the electronic device 400, the server 500 (e.g., the processor 510) may retrieve the content corresponding to the specific skin, and may provide the retrieved content to the electronic device 400.

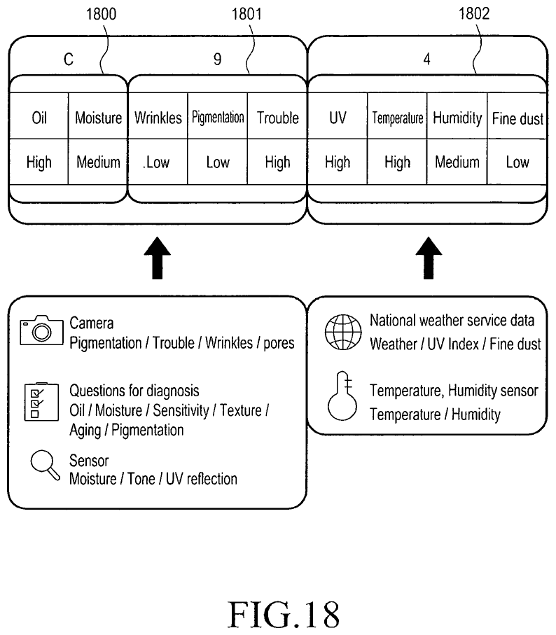

FIG. 18 illustrates an exemplary view showing a skin type according to various embodiments.

Referring to FIG. 18, the electronic device 400 (e.g., the processor 410) may analyze the skin of a user according to a first skin analysis item, such as wrinkles, pigmentation, trouble, pores, or the like, using the camera module 420, may analyze a user input received through the user interface for diagnosis questions according to a second skin analysis item, such as oil, moisture, sensitivity, texture, aging, pigmentation, or the like, or may analyze measurement data measured through the sensor module 430 according to a third skin analysis item, such as moisture, tone, UV reflection, or the like, thereby obtaining skin analysis information.

The electronic device 400 (e.g., the processor 410) may analyze weather data (e.g., weather, a UV index, fine dust concentration, or the like) provided from a weather information provider, such as the National Weather Service, according to a first environment analysis item, such as a UV index, temperature, humidity, fine dust concentration, or the like, or may perform analysis according to a second environment analysis item, such as temperature, humidity, or the like, using the sensor module 430 including a temperature sensor, a humidity sensor, and the like, thereby obtaining environment analysis information.

The electronic device 400 (e.g., the processor 410), based on rating configuration information on respective skin analysis items, may identify one or more ratings for respective skin analysis items corresponding to the skin analysis information, and, based on rating configuration information on respective environment analysis items, may identify the ratings for respective environment analysis items corresponding to the environment analysis information.

For example, the electronic device 400 (e.g., the processor 410), based on the rating configuration information on respective skin analysis items, may identify: the rating corresponding to the oil item (e.g., High); the rating corresponding to the moisture item (e.g., Medium); the rating corresponding to the wrinkle item (e.g., Low); the rating corresponding to the pigmentation item (e.g., Low); and the rating corresponding to the trouble item (e.g., High).

For example, the electronic device 400 (e.g., the processor 410), based on the rating configuration information on respective environment analysis items, may identify: the rating corresponding to the UV item (e.g., High); the rating corresponding to the temperature item (e.g., Medium); the rating corresponding to the humidity item (e.g., Medium); and the rating corresponding to the fine dust item (e.g., Low).

The electronic device 400 (e.g., the processor 410) may use at least one skin type table, and may determine the skin type corresponding to “Oil: High and Moisture: Medium” (1800), “Wrinkles: Low, Pigmentation: Low, and Trouble: High” (1801), and “UV index: High, Temperature: High, Humidity: Medium, and Fine dust concentration: Low” (1802). For example, the determined skin type may be “C94”.

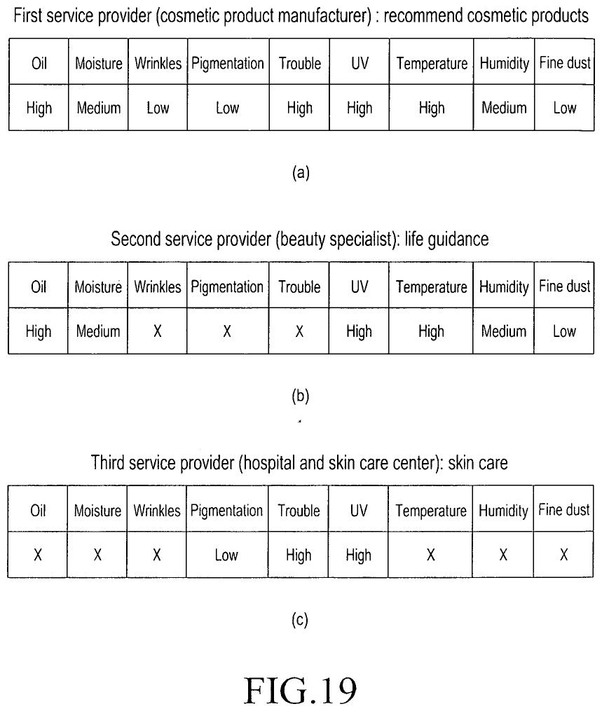

FIG. 19 illustrates exemplary views explaining an operation of selecting ratings for respective skin analysis items according to a service provider according to various embodiments.

Referring to FIG. 19, a service provider device 600 (e.g., the processor 610) may display, on the display 640, a first user interface for selecting ratings for the skin analysis items including oil, moisture, wrinkles, pigmentation, trouble, and the like and the environment analysis items including a UV index, temperature, humidity, fine dust concentration, and the like. When an input for selecting the ratings is received from a service provider through the first user interface, the service provider device 600 (e.g., the processor 610) may identify the skin type according to the input, may display information on the identified skin type (e.g., text, images, or the like), and may further display a second user interface for uploading content related to the identified skin type. When an input for selecting the content related to the identified skin type is received from the service provider through the second user interface, the service provider device 600 (e.g., the processor 610) may transmit the content selected by the input to the server 500. Here, the service provider may be a content provider for providing content according to the skin type of a user (e.g., a content receiver) of the electronic device 400.

In the case where the first service provider is a cosmetic product manufacturer, the service provider device 600 (e.g., the processor 610), as shown in FIG. 19A, may receive an input, such as “Oil: High, Moisture: Medium, Wrinkles: Low, Pigmentation: Low, Trouble: High, UV index: High, Temperature: High, Humidity: Medium, and Fine dust concentration: Low”, through the user interface. The service provider device 600 (e.g., the processor 610) may identify the skin type corresponding to the received input, and may display information on the identified skin type on the display 640.

In the case where the second service provider is a beauty specialist, the service provider device 600 (e.g., the processor 610), as shown in FIG. 19B, may receive an input, such as “Oil: High, Moisture: Medium, Wrinkles: X, Pigmentation: X, Trouble: X, UV index: High, Temperature: High, Humidity: Medium, and Fine dust concentration: Low”, through the user interface. For example, the input, such as “Wrinkles: X, Pigmentation: X, and Trouble: X” may mean that the ratings for the wrinkle, pigmentation, and trouble items have not been selected. In this case, the service provider device 600 (e.g., the processor 610) may identify the skin type corresponding to the ratings for the oil, moisture, UV index, temperature, humidity, and fine dust items, excluding the wrinkle, pigmentation, and trouble items the ratings of which have not been selected, and may display information on the identified skin type on the display 640.

In the case where the third service provider is a skin-related hospital and skin care center, the service provider device 600 (e.g., the processor 610), as shown in FIG. 19C, may receive an input, such as “Oil: X, Moisture: X, Wrinkles: X, Pigmentation: Low, Trouble: High, UV index: High, Temperature: X, Humidity: X, and Fine dust concentration: X”, through the user interface. For example, the input, such as “Oil: X, Moisture: X, Wrinkles: X, Temperature: X, Humidity: X, and Fine dust concentration: X” may mean that the ratings for the oil, moisture, wrinkle, temperature, humidity, and fine dust items have not been selected. In this case, the service provider device 600 (e.g., the processor 610) may identify the skin type corresponding to the ratings for the pigmentation, trouble, and UV index items, excluding the oil, moisture, wrinkle, temperature, humidity, and fine dust items the ratings of which have not been selected, and may display information on the identified skin type on the display 640.

Accordingly, the above service providers transmit the content related to at least one skin type to the server 500 using the service provider device 600, thereby allowing the user to conveniently measure the skin type according to the skin diagnosis and to easily receive the content related to the measured skin type.

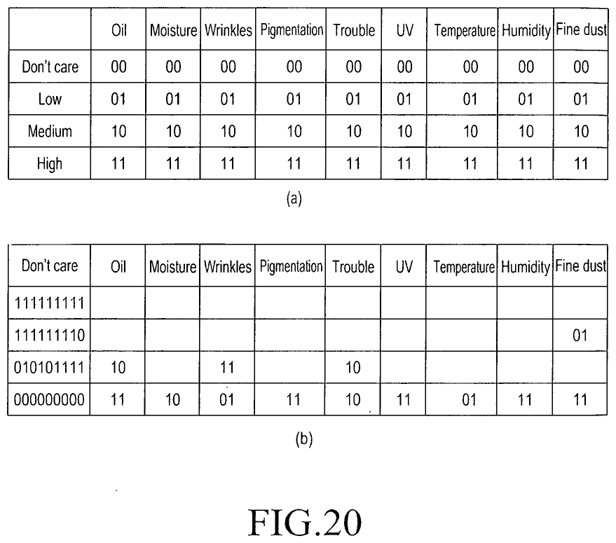

FIG. 20 illustrates exemplary views explaining skin rating information according to various embodiments.

Referring to FIG. 20A, the skin rating information may be generated as data of a total of 18 bits that are allocated by 2 bits for each rating according to skin analysis items and environment analysis items.

According to an embodiment, based on a bit information table expressed by two bits (e.g., 00, 01, 10, 11) corresponding to the ratings, such as “Don't care” (e.g., selection is not made by a service provider), High, Medium, and Low, according to skin analysis items and environment analysis items, such as oil, moisture, wrinkles, pigmentation, trouble, UV, temperature, humidity, and fine dust concentration, as shown in FIG. 20A, the skin analysis information may be represented in a 2-bit format.

For example, if the skin rating information is “Oil: High, Moisture: Medium, Wrinkles: Low, Pigmentation: Low, Trouble: High, UV index: High, Temperature: High, Humidity: Medium, and Fine dust concentration: Low”, the service provider device 600, based on the bit information table, may generate data in a 2-bit format such as “111001011111111001”.

For example, if the skin rating information is “Oil: High, Moisture: Medium, Wrinkles: X, Pigmentation: X, Trouble: X, UV index: High, Temperature: High, Humidity: Medium, and Fine dust concentration: Low”, the service provider device 600, based on the bit information table, may generate data in a 2-bit format such as “111000000011111001”.

For example, if the skin rating information is “Oil: X, Moisture: X, Wrinkles: X, Pigmentation: Low, Trouble: High, UV index: High, Temperature: X, Humidity: X, and Fine dust concentration: X”, the service provider device 600, based on the bit information table, may generate data in a 2-bit format such as “000000011111000000”.

According to various embodiments, in addition to one or more skin analysis items and environment analysis items (e.g., 9 analysis items), as shown in FIG. 20B, the skin rating information may further include a rating item (e.g., “Don't care”) indicating the presence or absence of the rating for each analysis item so as to be generated as data in a 9 to 27-bit format. The data on the rating item of the skin rating information may be configured as a total of 9 bits, wherein the respective analysis items are expressed as “0” when they have ratings (e.g., when the ratings have not been selected by the service provider) and are expressed as “1” when they have no rating (e.g., when the ratings have been selected by the service provider). Accordingly, the server 500 may identify the rating item included in the header of the content corresponding to the skin type, and may identify only the analysis items having the ratings and the ratings corresponding thereto, thereby identifying the skin rating information more quickly.

For example, if there is no rating for at least one skin analysis item and environment analysis item (e.g., 9 items), the skin rating information may be configured as data of 9 bits (e.g., “111111111”) indicating that there is no rating for 9 analysis items corresponds to the rating item.

For example, if there is a rating for the fine dust concentration item, such as “Fine dust concentration: Low”, among one or more skin analysis items and environment analysis items, the skin rating information may be configured as data of a total of 11 bits (e.g., “11111111001”) including data of 9 bits (e.g., “111111110”) indicating that there is a rating for the fine dust concentration item of 9 analysis items corresponding to the rating item and data of 2 bits (e.g., “01”) indicating the rating corresponding to the fine dust concentration item.

For example, if there are ratings for the oil, wrinkle, and trouble items, such as “Oil: Medium, Wrinkles: High, and Trouble: Medium”, among one or more skin analysis items and environment analysis items, the skin rating information may be configured as data of a total of 15 bits (e.g., “010101111101110”) including data of 9 bits (e.g., “010101111”) indicating that there are ratings for the oil, wrinkles, and trouble items of 9 analysis items corresponding to the rating item and data of 6 bits (e.g., “101110”) indicating the ratings corresponding to the oil, wrinkles, and trouble items.

For example, if there are ratings for the oil, moisture, wrinkles, pigmentation, trouble, UV index, temperature, humidity, and fine dust concentration items, such as “Oil: High, Moisture: Medium, Wrinkles: Low, Pigmentation: High, Touble: Medium, UV index: High, Temperature: Medium, Humidity: High, and Fine dust concentration: High”, among one or more skin analysis items and environment analysis items, the skin rating information may be configured as data of a total of 27 bits (e.g., “000000900111001111011011111”) including data of 9 bits (e.g., “000000000”) indicating that there are ratings for 9 analysis items corresponding to the rating item and data of 18 bits (e.g., “111001111011011111”) corresponding to the oil, moisture, wrinkles, pigmentation, trouble, UV index, temperature, humidity, and fine dust concentration items.

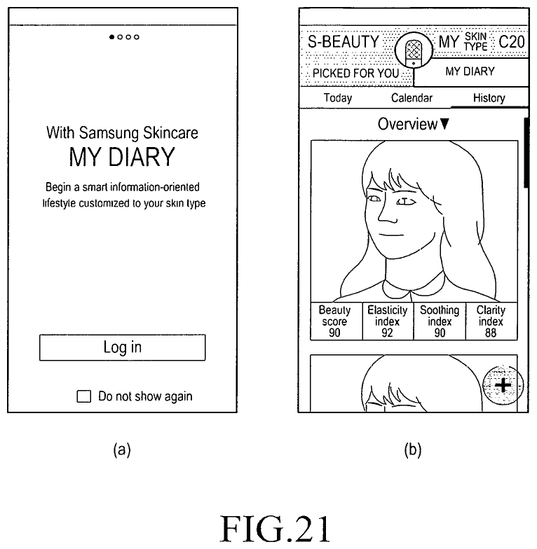

FIGS. 21 to 36 illustrate exemplary views for describing the operation of a skin care application to provide content for each skin type in an electronic device according to various embodiments.

FIG. 21 illustrates exemplary views of the execution screen of a skin care application that provides content for each skin type according to various embodiments.

Referring to FIG. 21, when an input for executing a skin care application for providing content for each skin type is received through the input device 470, the electronic device 400 (e.g., the processor 410) may execute a skin care application, and may display a user login screen of the skin care application, as shown in FIG. 21A. When an input for login is received through the input device 470, the electronic device 400 (e.g., the processor 410) may display a login input screen for inputting an ID/password.

When the ID/password is input through the login input screen, the electronic device 400 (e.g., the processor 410) may display a start screen of the skin care application as shown in FIG. 21B. The electronic device 400 (e.g., the processor 410) may display, as a default start screen, a history screen (e.g., a history information display screen of the skin care application) showing a history of using the skin care application by the user. The history screen may include a face image of the user and past records of beauty scores indicating the overall skin condition, an elasticity index indicating the elasticity of the skin, a soothing index indicating the degree of trouble of the skin, a clarity index indicating the transparency of the skin, and the like.

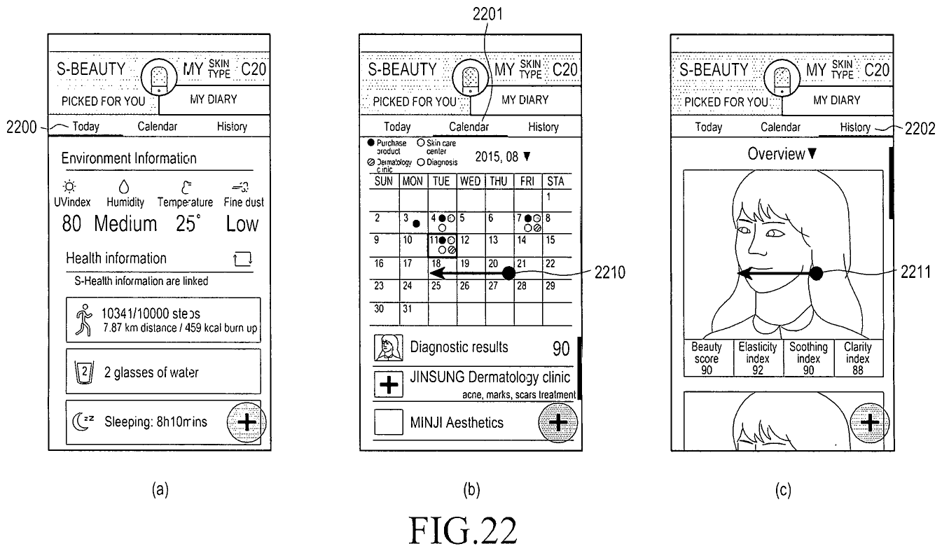

FIG. 22 illustrates various exemplary screens for a start screen of a skin care application according to various embodiments.

Referring to FIG. 22, the start screen may include a first start screen (e.g., Today 2200) indicating today's environment state and a user's health condition, a second start screen (e.g., Calendar 2201) indicating the schedule for the user to purchase skin care products, to visit a skin care center, to see a dermatologist, to perform skin diagnosis, and the like using the skin care application, and a third start screen (e.g., History 2202) indicating a history of using the skin care application by the user.

As shown in FIG. 22A, the first start screen may include an area showing environment information including a UV index, humidity, temperature, and fine dust concentration of today and an area showing health information including the amount of calorie consumption, the amount of moisture intake, and sleeping time of the user.

The second start screen, as shown in FIG. 22B, may include an area showing a schedule, such as purchase of skin-related products, a visit to a skin care center, a visit to a dermatology, skin diagnosis, and the like for a month in August 2015, and an area showing a diagnosis result, dermatology information, skin care center information, and the like.

The third start screen, as shown in FIG. 22C, may include areas showing the face image of a user taken for skin diagnosis and areas showing past records of a beauty score, an elasticity index, a soothing index, a clarity index, and the like.

When a touch-and-drag input 2210 or 2211 (e.g., a swipe) is received through the input device 470 (e.g., a touch screen), the electronic device 400 may switch from the first start screen to the second start screen or from the second start screen to the third start screen, thereby display the screen.

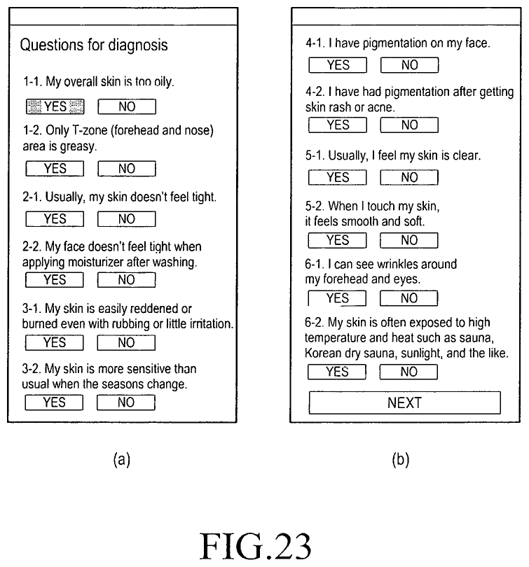

FIG. 23 illustrates exemplary views of a user interface for skin diagnosis of a user according to various embodiments.

Referring to FIG. 23, the electronic device 400 (e.g., the processor 410) may display, on the display 450, a user interface including queries for skin diagnosis of a user (e.g., questions for skin diagnosis 1-1 to 6-2).

When a request for determining the skin type of the user is received through the input device 470, the electronic device 400 (e.g., the processor 410) may display a first diagnosis screen including the queries (e.g., 1-1, 1-2, 2-1, 2-2, 3-1, and 3-2) for one or more skin analysis items as shown in FIG. 23A. When a user input on the first diagnosis screen is received, the electronic device 400 (e.g., the processor 410) may display a second diagnosis screen including the queries (e.g., 4-1, 4-2, 5-1, 5-2, 6-1, and 6-2) for one or more skin analysis items as shown in FIG. 23B. When a user input on the second diagnosis screen is received, the electronic device 400 (e.g., the processor 410) may perform an operation for diagnosing the skin of the user using the camera module 420.

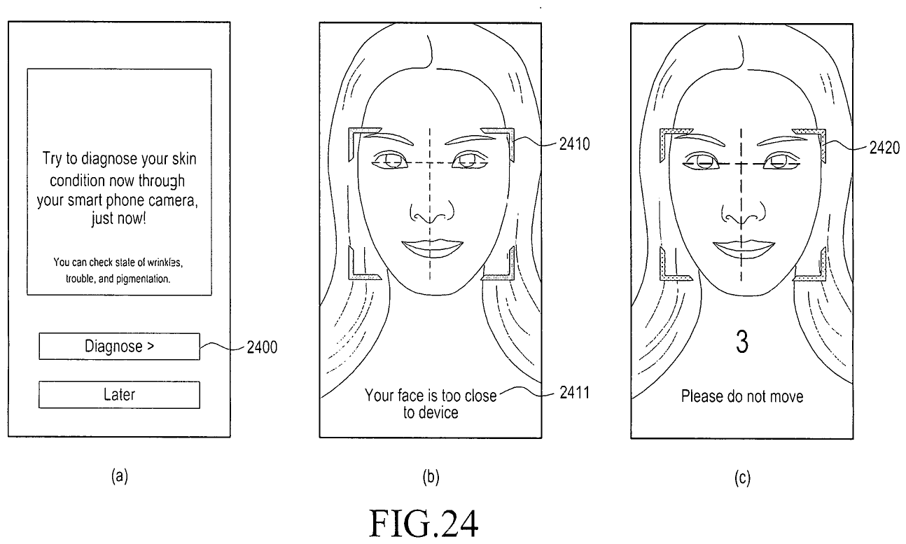

FIG. 24 illustrates exemplary views of a user interface for skin diagnosis of a user using a camera module according to various embodiments

Referring to FIG. 24, the electronic device 400 (e.g., the processor 410) may display a diagnosis start screen for diagnosing a user's skin condition using the camera module 420 as shown in FIG. 24A. When a user input for selecting a first object 2400 for starting the diagnosis is received, the electronic device 400 (e.g., the processor 410) may display a photographing standby screen for photographing the face of a user on the display 450 as shown in FIG. 24B.

The electronic device 400 (e.g., the processor 410) may display a guide area 2410 for adjusting the size of the face on the photographing standby screen, and if the size of the recognized face area is larger or smaller than the size of the face area predetermined for face recognition, the electronic device 400 may display, on the display 450, a second object 2411 for adjusting the distance between the user and the camera module 420 (e.g., an information message such as “face is too close”).

If the size of the recognized face area matches the size of the face area predetermined for face recognition, the electronic device 400 (e.g., the processor 410) may display, on the display 450, a third object 2420 to indicate that the size of the recognized face area is suitable for face recognition (e.g., changing the color of the first object 2410) as shown in FIG. 24C.

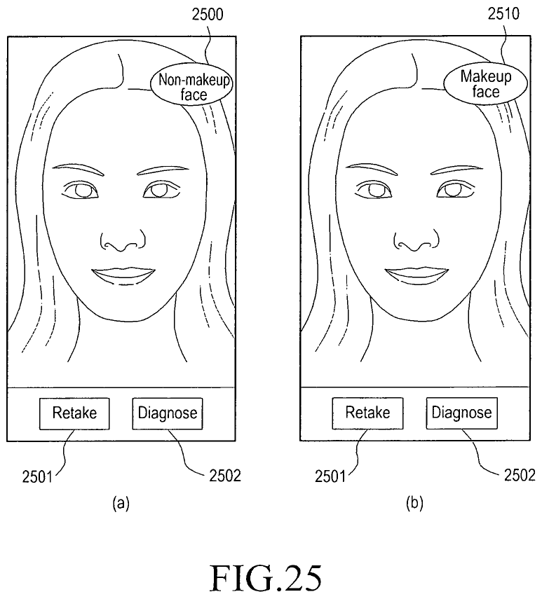

FIG. 25 illustrates exemplary views for determining whether a photographed image represents a makeup face or a non-makeup face according to various embodiments.

Referring to FIG. FIG. 25, the electronic device 400 (e.g., the processor 410) may display a photographed face image as shown in FIG. 25A, and may display a fourth object 2500 indicating a non-makeup face on the upper portion of the photographed face image. When a user input (e.g., a touch input, a gesture input, a fingerprint input, a voice input, or the like) for selecting the displayed fourth object 2500 is received, the electronic device 400 (e.g., the processor 410) may display a fifth object 2501 for photographing the face again (e.g., an icon, a button, an area, or an image for “Retake”) and a sixth object 2502 for skin condition analysis for the photographed face image (e.g., an icon, a button, an area, or an image for “Analyze”). When a user input (e.g., a touch input, a gesture input, a fingerprint input, a voice input, or the like) for selecting the fifth object 2501 is received, the electronic device 400 (e.g., the processor 410) may perform retake. When a user input for selecting the sixth object 2502 is received, the electronic device 400 (e.g., the processor 410) may analyze the skin condition for the face image that has been set as a non-makeup face.

When a user input for reselecting the fourth object 2500 is received, the electronic device 400 (e.g., the processor 410), as shown in 25B, may display a seventh object 2510 indicating a makeup face on the upper portion of the displayed face image, and may display the fifth object 2501 (e.g., an icon, a button, an area, or an image for “Retake”) and the sixth object 2502 (an icon, a button, an area, or an image for “Analyze”). When a user input for selecting the fifth object 2501 is received, the electronic device 400 (e.g., the processor 410) may perform retake. When a user input for selecting the sixth object 2502 is received, the electronic device 400 (e.g., the processor 410) may analyze the skin condition for the face image that has been set as a makeup face.

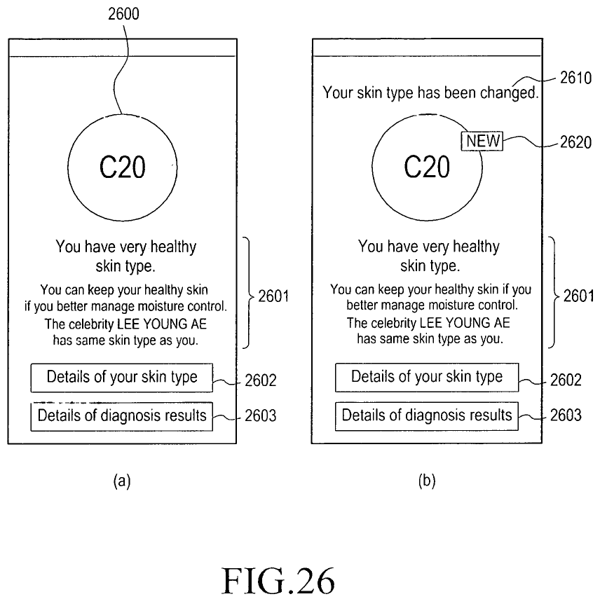

FIG. 26 illustrates exemplary views showing information on a determined skin type according to various embodiments.

Referring to FIG. 26, based on a user input received through a user interface for skin diagnosis and skin analysis information obtained by analyzing the photographed face image, the electronic device 400 (e.g., the processor 410) may determine a skin type, and may display information on the determined skin type as shown in FIG. 26A.

For example, a screen displaying the information on the skin type may include an eighth object 2600 indicating the skin type (e.g., text, an image, or the like such as C20), a ninth object 2601 (e.g., text, image, or the like) indicating a description of the skin type, a tenth object 2602 for displaying detailed information on the skin type (e.g., an icon, a button, an area, or an image of “Details of skin type”), and an eleventh object 2603 for displaying detailed information on the skin diagnosis (e.g., an icon, a button, an area, or an image of “Details of diagnosis result”).

For example, if there is history information on the skin type of a user, which has been diagnosed in the past, the electronic device 400 may compare the previous skin type with the current skin type. If the skin type has been changed, the electronic device 400, as shown in FIG. 26B, may display a twelfth object 2610 indicating information on the changed skin type (e.g., a notice of a change in the skin type, such as “skin type has been changed”, displayed on the screen) and a thirteenth object 2620 indicating the new skin type (e.g., an icon or image of “New”).

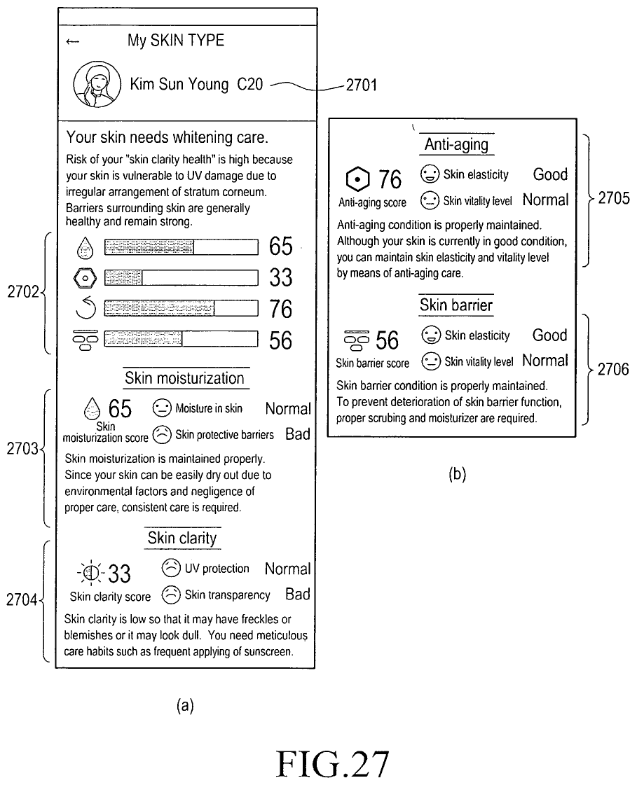

FIG. 27 illustrates exemplary views for showing detail information on the skin type according to various embodiments.

Referring to FIG. 27, the electronic device 400 (e.g., the processor 410), as shown in FIG. 27A, may display a screen including an area 2701 indicating information on the skin type, an area 2702 representing numeric values for one or more skin analysis items (e.g., oil, moisture, wrinkles, pigmentation, trouble, or the like), an area 2703 indicating information on the skin moisturization, and an area 2704 indicating information on the skin brightness. The electronic device 400 may display a screen that further includes an area 2705 indicating information on anti-aging and an area 2706 indicating information on the skin barrier, as shown in FIG. 27B.

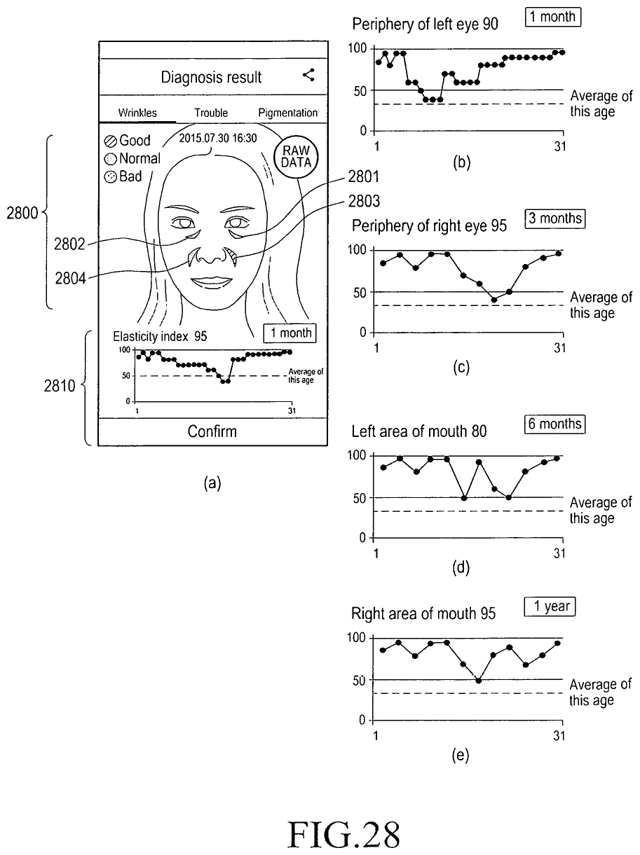

FIGS. 28 to 30 illustrate exemplary views for showing detailed information on skin diagnosis results according to various embodiments.

FIG. 28 illustrates exemplary views of skin diagnosis results according to a wrinkle item in various embodiments.

Referring to FIG. 28, the electronic device 400 (e.g., the processor 410) may display skin analysis information obtained by analyzing the skin corresponding to the area on the periphery of the left eye(“left eye-peripheral area”), the area on the periphery of the right eye(“right eye-peripheral area”), the area on the periphery of the left side of a mouth(“left mouth-peripheral area”), and the area on the periphery of the right side of a mouth(“right mouth-peripheral area”) in the face area of a user according to a wrinkle item.

For example, the electronic device 400 (e.g., the processor 410) may display a screen including an area 2800 showing the face image of a user and an area 2810 showing skin analysis information obtained by analyzing the skin corresponding to the left eye-peripheral area 2801, the right eye-peripheral area 2802, the left mouth-peripheral area 2803, and the right mouth-peripheral area 2804 according to the wrinkle item, as shown in FIG. 28A. The electronic device 400 (e.g., the processor 410) may display diagnosis information (e.g., good, normal, or bad) on the left eye-peripheral area 2801, the right eye-peripheral area 2802, the left mouth-peripheral area 2803, and the right mouth-peripheral area 2804 in different colors (e.g., green, blue, or red).

The skin analysis information obtained by analyzing the skin corresponding to the left eye-peripheral area 2801 may be represented as shown in FIG. 28B, the skin analysis information obtained by analyzing the, skin according to the right eye-peripheral area 2802 may be represented as shown in FIG. 28C, the skin analysis information obtained by analyzing the skin corresponding to the left mouth-peripheral area 2803 may be represented as shown in FIG. 28D, and the skin analysis information obtained by analyzing the skin corresponding to the right mouth-peripheral area 2804 may be represented as shown in FIG. 28E. The skin analysis information may be displayed in a graph form. The horizontal axis of the displayed graph may represent the dates on which skin diagnosis has been performed in a month, and the vertical axis of the graph may represent a value (e.g., a value between 0 and 100) obtained by quantifying the skin diagnosis result for the wrinkle item.

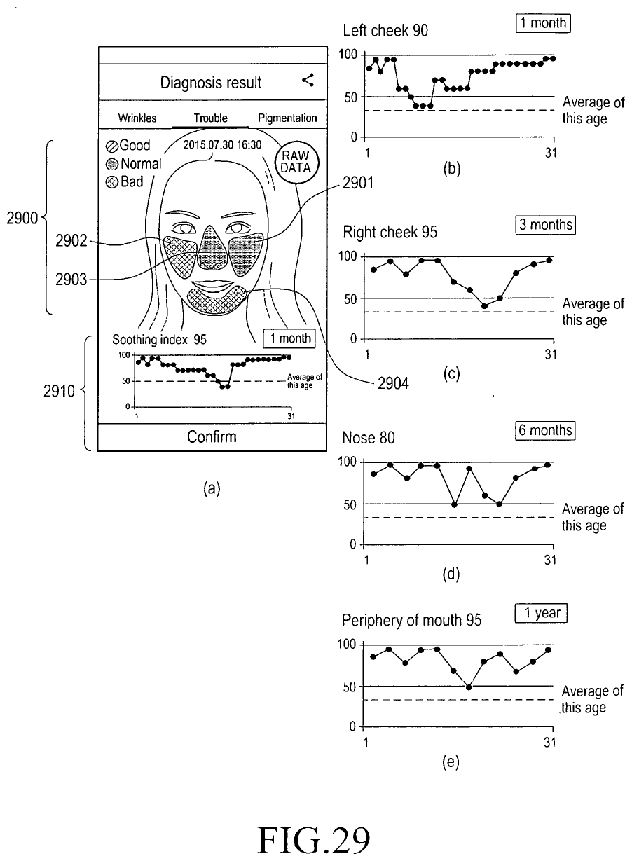

FIG. 29 illustrates exemplary views showing skin diagnosis results according to a trouble item according to various embodiments.

Referring to FIG. 29, the electronic device 400 (e.g., the processor 410) may display skin analysis information obtained by analyzing the skin corresponding to the left cheek area, the right cheek area, the nose area, and the mouth-peripheral area in the lace area of a user according to a trouble item.

For example, the electronic device 400 (e.g., the processor 410) may display a screen including an area 2900 showing the face image of a user and an area 2910 indicating skin analysis information obtained by analyzing the skin corresponding to the left cheek area 2901, the right cheek area 2902, the nose area 2903, and the mouth-peripheral area 2904 according to the trouble item, as shown in FIG. 29A. The electronic device 400 (e.g., the processor 410) may display diagnosis results (e.g., good, normal, or bad) on the skin corresponding to the left cheek area 2901, the right cheek area 2902, the nose area 2903, and the mouth-peripheral area 2904 in different colors (e.g., green, blue, or red).

The skin analysis information obtained by analyzing the skin corresponding to the left cheek area 2901 may be represented as shown in FIG. 29B, and the skin analysis information obtained by analyzing the skin corresponding to the right cheek area 2902 may be represented as shown in FIG. 29C. The skin analysis information obtained by analyzing the skin corresponding to the nose area 2903 may be represented as shown in FIG. 29D, and the skin analysis information obtained by analyzing the skin corresponding to the mouth-peripheral area 2904 may be represented as shown in FIG. 29E. The skin analysis information may be displayed in a graph form. The horizontal axis of the displayed graph may represent the dates on which skin diagnosis has been performed in a month, and the vertical axis of the graph may represent a value (e.g., a value between 0 and 100) obtained by quantifying the skin diagnosis result for the trouble item.

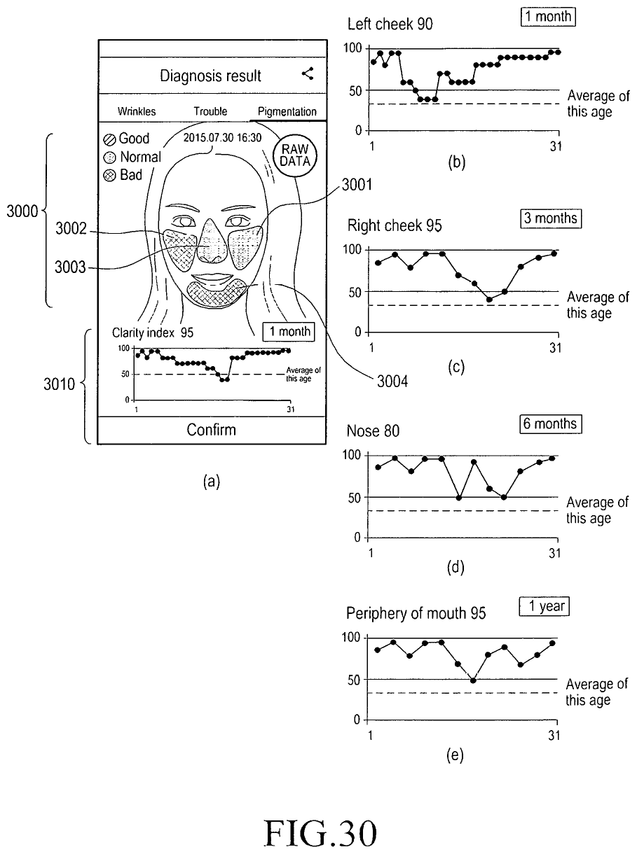

FIG. 30 illustrates exemplary views showing skin diagnosis results according to a pigmentation item according to various embodiments.

Referring to FIG. 30, the electronic device 400 (e.g., the processor 410) may display skin analysis information obtained by analyzing skin corresponding to the left cheek area, the right check area, the nose area, and the mouth-peripheral area in the face area of a user according to a pigmentation item.

For example, the electronic device 400 (e.g., the processor 410) may display a screen including an area 3000 showing the face image of a user and an area 3010 indicating skin analysis information obtained by analyzing the skin corresponding to the left cheek area 3001, the right cheek area 3002, the nose area 3003, and the mouth-peripheral area 3004 according to the pigmentation item, as shown in FIG. 30A. The electronic device 400 (e.g., the processor 410) may display diagnosis results (e.g., good, normal, or bad) on the skin corresponding to the left cheek area 3001, the right cheek area 3002, the nose area 3003, and the mouth-peripheral area 3004 in different colors (e.g., green, blue, or red).

The skin analysis information obtained by analyzing the skin corresponding to the left cheek area 3001 may be represented as shown in FIG. 30B, and the skin analysis information obtained by analyzing the skin corresponding to the right cheek area 3002 may be represented as shown in FIG. 30C. The skin analysis information obtained by analyzing the skin corresponding to the nose area 3003 may be represented as shown in FIG. 30D, and the skin analysis information obtained by analyzing the skin around the mouth-peripheral area 3004 may be represented as shown in FIG. 30E. The skin analysis information may be displayed in a graph form. The horizontal axis of the displayed graph may represent the dates on which skin diagnosis has been performed in a month, and the vertical axis of the graph may represent a value (e.g., a value between 0 and 100) obtained by quantifying the skin diagnosis result for the pigmentation item.

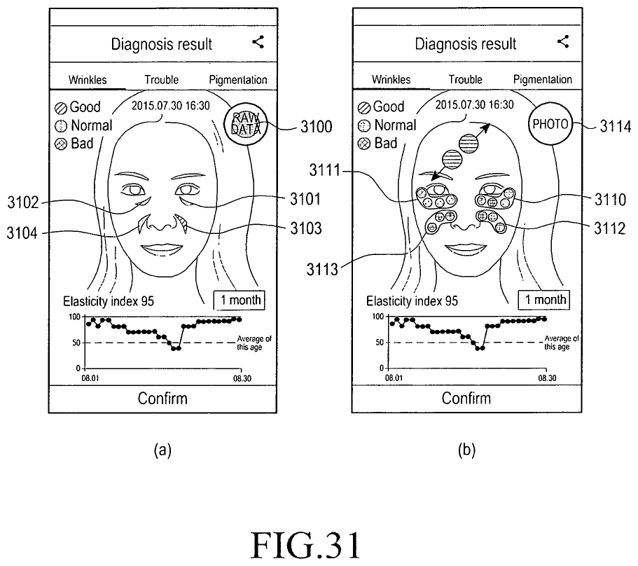

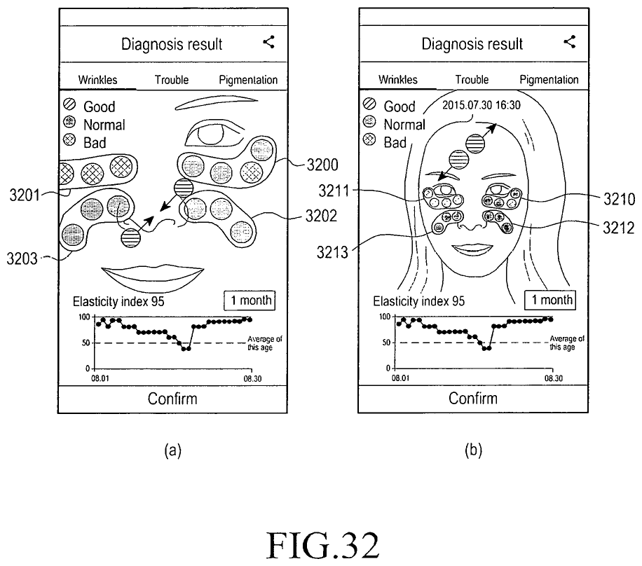

FIGS. 31 and 32 illustrate exemplary views for showing detailed skin analysis information on a specific area by enlarging a face image according to various embodiments.

Referring to FIG. 31, the electronic device 400 (e.g., the processor 410) may display fourteenth object 3100 (e.g., an icon, image, area, or the like of “RAW DATA”) for showing detailed diagnosis information on the skin corresponding to the left eye-peripheral area 3101, the right eye-peripheral area 3102, the left mouth-peripheral area 3103, and the right mouth-peripheral area 3104 of a face area as shown in FIG. 31A. When a user input (e.g., a touch input, a gesture input, a fingerprint input, a voice input, or the like) for selecting the fourteenth object 3100 is received, the electronic device 400 (e.g., the processor 410) may display detailed diagnosis information on the skin corresponding to the left eye-peripheral area 3110, the right eye-peripheral area 3111, the left mouth-peripheral area 3112, and the right mouth-peripheral area 3113 as shown in FIG. 31B. The electronic device 400 (e.g., the processor 410) may further display a fifteenth object 3114 for showing diagnosis information on the skin corresponding to the left eye-peripheral area, the right eye-peripheral area, the left mouth-peripheral area, and the right mouth-peripheral area of the face image.

Referring to FIG. 32, when a user input (e.g., a touch input, a touch-and-drag input, a two-touch input, a gesture input, a fingerprint input, a voice input, or the like) for enlarging the face image is received, the electronic device 400 (e.g., the processor 410) may enlarge the face image, thereby displaying the detailed diagnosis information on the skin corresponding to the left eye-peripheral area 3200, the right eye-peripheral area 3201, the left mouth-peripheral area 3202, and the right mouth-peripheral area 3203 as shown in FIG. 32A.

When a user input (e.g., a touch input, a touch-and-drag input, a two-touch input, a gesture input, a fingerprint input, a voice input, or the like) for reducing the face image is received, the electronic device 400 may reduce the face image, thereby displaying the detailed diagnosis information on the skin corresponding to the left eye-peripheral area 3210, the right eye-peripheral area 3211, the left mouth-peripheral area 3212, and the right mouth-peripheral area 3213 as shown in FIG. 32B.

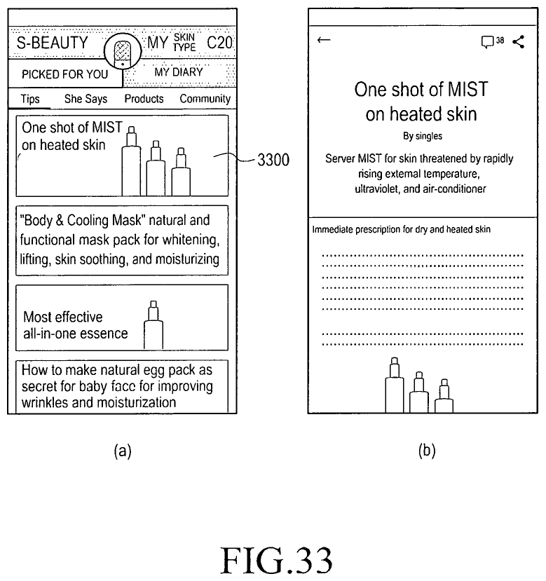

FIGS. 33 to 36 illustrate exemplary views for displaying a variety of content corresponding to the skin type according to various embodiments.

Referring to FIGS. 33 to 36, the electronic device 400 (e.g., the processor 410) may display content (e.g., skin care tips, skin care information proposed by a role model, cosmetic product information, community information, or the like) related to the determined skin type on the display 450.

Referring to FIG. 33, the electronic device 400 (e.g., the processor 410) may display a screen showing information on skin care tips for the determined skin type as shown in FIG. 33A. According to an embodiment, the electronic device 400 (e.g., the processor 410) may make a request to the server 500 for the content corresponding to a specific skin type (e.g., C20), and may display the content (e.g., text, images, videos, or a webpage link for the skin care tips corresponding to C20) received from the server 500. The content may include content matched the specific skin type by at least one service provider.

For example, when a user input (e.g., a touch input, a gesture input, a fingerprint input, a voice input, or the like) for selecting a sixteenth object 3300 for displaying first content corresponding to the skin care tips is received, the electronic device 400 (e.g., the processor 410) may display detailed information on the first content (e.g., detailed information on a specific skin care tip corresponding to C20) as shown in FIG. 33B.

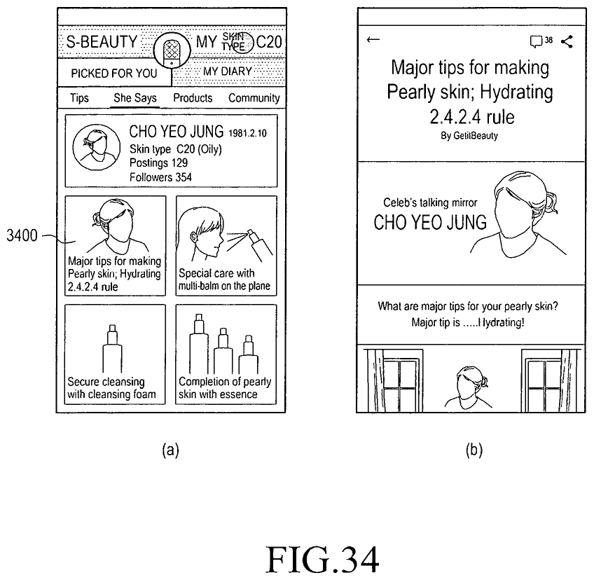

Referring to FIG. 34, the electronic device 400 (e.g., the processor 410) may display a screen showing skin care information proposed by a role model for the determined skin type as shown in FIG. 34A. According to an embodiment, the electronic device 400 (e.g., the processor 410) may make a request to the server 500 for the content corresponding to a specific skin type (e.g., C20), and may display the content (e.g., text, images, videos, or a webpage link for the skin care information proposed by the role model corresponding to C20) received from the server 500.

For example, when a user input (e.g., a touch input, a gesture input, a fingerprint input, a voice input, or the like) for selecting a seventeenth object 3400 for displaying second content corresponding to the skin care information proposed by the role model is received, the electronic device 400 (e.g., the processor 410) may display detailed information on the second content (e.g., skin care information proposed by a specific role model corresponding to C20) as shown in FIG. 34B.

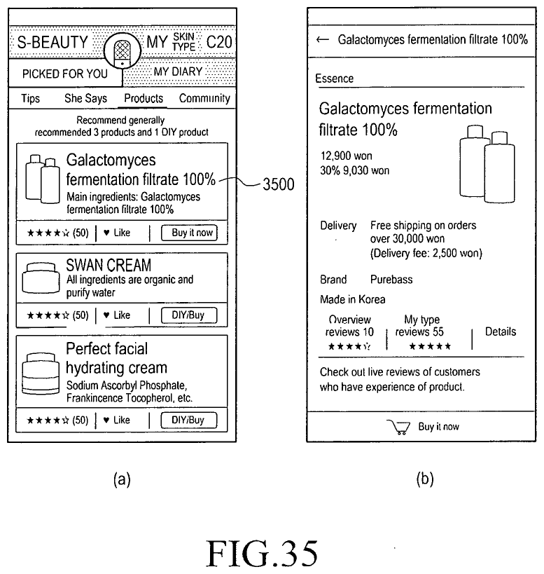

Referring to FIG. 35, the electronic device 400 (e.g., the processor 410) may display a screen showing cosmetic product information for the determined skin type as shown in FIG. 35A. According to an embodiment, the electronic device 400 (e.g., the processor 410) may make a request to the server 500 for the content corresponding to a specific skin type (e.g., C20), and may display the content (e.g., text, images, videos, or a webpage link for cosmetic product information corresponding to C20) received from the server 500.

For example, when a user input (e.g., a touch input, a gesture input, a fingerprint input, a voice input, or the like) for selecting an eighteenth object 3500 for displaying third content corresponding to the cosmetic product information is received, the electronic device 400 (e.g., the processor 410) may display detailed information on the third content (e.g., specific cosmetic product information corresponding to C20) as shown in FIG. 35B.

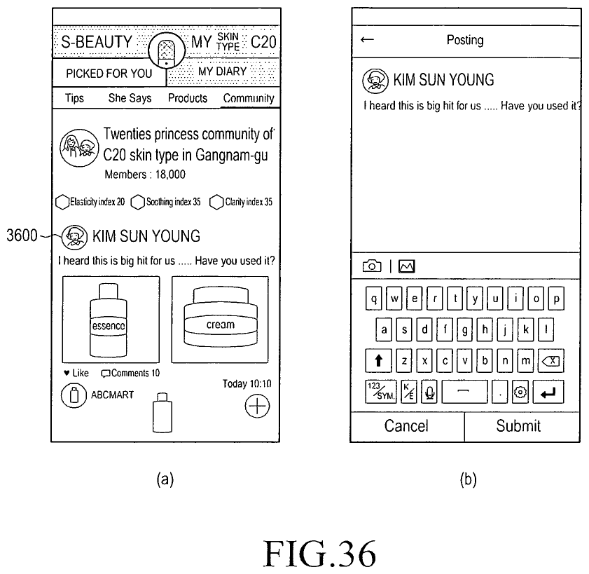

Referring to FIG. 36, the electronic device 400 (e.g., the processor 410) may display a screen showing community information on the determine skin type (e.g., information on users of the same skin type) as shown in FIG. 36A. Here, the community information may include personal information on members of a specific skin type constituting the community, shared content thereof (e.g., articles, pictures, videos, or the like), and the like.

According to an embodiment, the electronic device 400 (e.g., the processor 410) may make a request to the server 500 for the content corresponding to a specific skin type (e.g., C20), and may display the content (e.g., text, images, videos, or a webpage link for the community information corresponding to C20) received from the server 500.

For example, when a user input (e.g., a touch input, a gesture input, a fingerprint input, a voice input, or the like) for selecting a nineteenth object 3600 for displaying fourth content corresponding to the community information is received, the electronic device 400 (e.g., the processor 410) may display detailed information on the fourth content (e.g., the community information corresponding to C20) as shown in FIG. 36B.

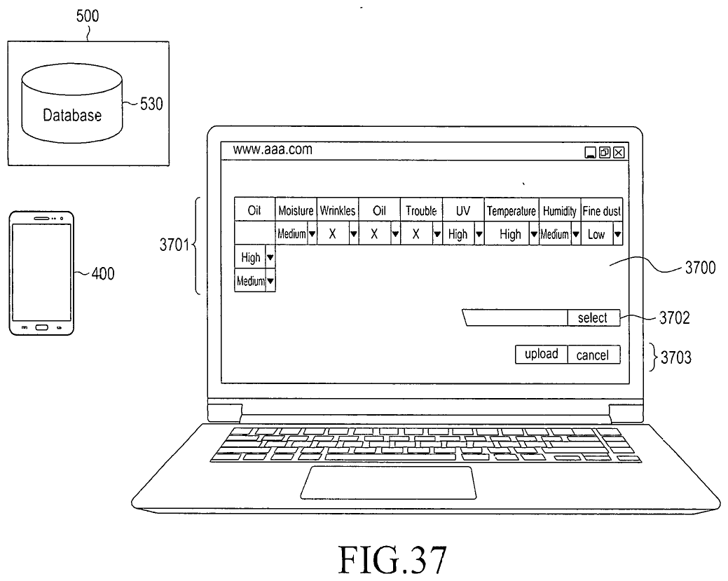

FIG. 37 illustrates an exemplary view of a user interface for selecting content according to the skin type in a service provider device according to various embodiments.

Referring to FIG. 37, the service provider device 600 (e.g., the processor 610) may display, on the display 640, a user interface 3700 including an area 3701 for selecting skin rating information according to skin analysis items and environment analysis items, an area 3702 for selecting the content corresponding to the selected skin rating information, and an area 3703 for uploading the content.

For example, a user input (e.g., a key input, a mouse cursor input, a touch input, or the like) for selecting ratings, such as High, Medium, and Low or High and Low, is received to correspond to oil, moisture, wrinkle, pigmentation, trouble, UV index, temperature, humidity, and fine dust concentration items, the service provider device 600 (e.g., the processor 610) may store, in the memory 630, skin rating information (e.g., “Oil: High, Moisture: Medium, Wrinkles: X, Pigmentation: X, Trouble: X, UV index: High, Temperature: High, Humidity: Medium, and Fine dust concentration: Low”) including the ratings selected to correspond to the oil, moisture, wrinkle, pigmentation, trouble, UV index, temperature, humidity, and fine dust concentration items. When a user input (e.g., a key input, a mouse cursor input, a touch input, or the like) for selecting the content corresponding to the skin rating information is received, the service provider device 600 (e.g., the processor 610) may include the skin rating information in the header of the selected content, thereby transmitting the same to the server 500.

The server 500 (e.g., the processor 510) may receive the content from the service provider device 600, and may match the received content with the skin type corresponding to the skin rating information included in the header of the content, thereby storing the same in the database 530. When a request for the content corresponding to a first skin type is received from the electronic device 400, the server 500 (e.g., the processor 510) may retrieve the content corresponding to the first skin type from the database 530, and may transmit the retrieved content to the electronic device 400.

As described above, various embodiments of the present disclosure provide an electronic device for providing content according to mobile-based measurement of a skin type, thereby allowing a user to conveniently measure a skin type according to skin diagnosis and easily obtaining content related to the measured skin type.

The term “module” as used herein may, for example, mean a unit including one of hardware, software, and firmware or a combination of two or more of them. The “module” may be interchangeably used with, for example, the term “unit”, “logic”, “logical block”, “component”, or “circuit”. The “module” may be a minimum unit of an integrated component element or a part thereof. The “module” may be a minimum unit for performing one or more functions or a part thereof. The “module” may be mechanically or electronically implemented. For example, the “module” according to the present disclosure may include at least one of an Application-Specific Integrated Circuit (ASIC) chip, a Field-Programmable Gate Arrays (FPGA), and a programmable-logic device for performing operations which has been known or are to be developed hereinafter.

According to various embodiments, at least some of the devices (for example, modules or functions thereof) or the method (for example, operations) according to the present disclosure may be implemented by a command stored in a computer-readable storage medium in a programming module form. The instruction, when executed by a processor (e.g., the processor 120), may cause the one or more processors to execute the function corresponding to the instruction. The computer-readable storage medium may, for example, be the memory 130.

The computer readable recoding medium may include a hard disk, a floppy disk, magnetic media (e.g., a magnetic tape), optical media (e.g., a Compact Disc Read Only Memory (CD-ROM) and a Digital Versatile Disc (DVD)), magneto-optical media (e.g., a floptical disk), a hardware device (e.g., a Read Only Memory (ROM), a Random Access Memory (RAM), a flash memory), and the like. In addition, the program instructions may include high class language codes, which can be executed in a computer by using an interpreter, as well as machine codes made by a compiler. The aforementioned hardware electronic device may be configured to operate as one or more software modules in order to perform the operation of the present disclosure, and vice versa.

The programming module according to the present disclosure may include one or more of the aforementioned components or may further include other additional components, or some of the aforementioned components may be omitted. Operations executed by a module, a programming module, or other component elements according to various embodiments of the present disclosure may be executed sequentially, in parallel, repeatedly, or in a heuristic manner. Furthermore, some operations may be executed in a different order or may be omitted, or other operations may be added. Various embodiments disclosed herein are provided merely to easily describe technical details of the present disclosure and to help the understanding of the present disclosure, and are not intended to limit the scope of the present disclosure. Therefore, it should be construed that all modifications and changes or modified and changed forms based on the technical idea of the present disclosure fall within the scope of the present disclosure.

1. An electronic device comprising:
a display; and
a processor, wherein the processor is configured to:
obtain skin diagnosis information indicating a skin condition of a user;
determine a skin type of the user, based on the skin diagnosis information; and
display information on the determined skin type and content corresponding to the determined skin type on the display.

2. The electronic device of claim 1, wherein the processor is configured to:
display, on the display, a user interface for receiving replies to queries predetermined for skin diagnosis from the user; and
when a user input is received through the user interface, analyze the received user input according to at least one skin analysis item, thereby obtaining first skin analysis information.

3. The electronic device of claim 2, further comprising at least one of a camera module and a sensor module,
wherein the processor is configured to analyze the skin of the user according to at least one skin analysis item using at least one of the camera module and the sensor module, thereby obtaining second skin analysis information.

4. The electronic device of claim 3, wherein the processor is configured to: based on rating configuration information on respective skin analysis items in which ratings are set to correspond to a plurality of threshold ranges for one or more skin analysis items, obtain skin rating information corresponding to at least one piece of the first skin analysis information and the second skin analysis information; and determine a skin type corresponding to the obtained skin rating information.

5. The electronic device of claim 4, further comprising a memory configured to store content corresponding to a plurality of skin types,
wherein the processor is configured to: retrieve the content corresponding to the determined skin type; and display information on the determined skin type and the retrieved content on the display.

6. The electronic device of claim 4, wherein the processor is configured to:
transmit a request for the content corresponding to the determined skin type to the server; and
when the content corresponding to the determined skin type is received from the server, display information on the determined skin type and the retrieved content on the display.

7. The electronic device of claim 6, wherein the processor is configured to:
based on the rating configuration information on respective skin analysis items in which ratings are set to correspond to a plurality of threshold ranges for one or more skin analysis items, obtain skin rating information corresponding to the first skin analysis information; and
determine a skin type corresponding to the obtained skin rating information.

8. A method for providing content according to a skin type in an electronic device, the method comprising:
obtaining skin diagnosis information indicating a skin condition of a user;
determining a skin type of the user, based on the skin diagnosis information; and
displaying information on the determined skin type and content corresponding to the determined skin type on display.

9. The method of claim 8, wherein the obtaining of skin diagnosis information comprises:
displaying a user interface for receiving replies to queries predetermined for skin diagnosis from the user; and
when a user input is received through the user interface, analyzing the received user input according to at least one skin analysis item, thereby obtaining first skin analysis information.

10. The method of claim 9, wherein the obtaining of skin diagnosis information comprises analyzing the skin of the user according to at least one skin analysis item using at least one of a camera module and a sensor module, thereby obtaining second skin analysis information.

11. The method of claim 10, wherein the determining of the skin type comprises:
based on rating configuration information on respective skin analysis items in which ratings are set to correspond to a plurality of threshold ranges for one or more skin analysis items, obtaining skin rating information corresponding to at least one piece of the first skin analysis information and the second skin analysis information; and
determining a skin type corresponding to the obtained skin rating information.

12. The method of claim 11, wherein the displaying of the information on the determined skin type and the content corresponding to the determined skin type on a display comprises:
retrieving content corresponding to the determined skin type from the content stored to correspond to a plurality of skin types; and
displaying information on the determined skin type and the retrieved content on the display.

13. The method of claim 8, wherein the obtaining of skin diagnosis information comprises:
displaying a user interface for receiving, from the user, replies to queries predetermined for skin diagnosis; and
when a user input is received through the user interface, analyzing the received user input according to at least one skin analysis item, thereby obtaining first skin analysis information.

14. The method of claim 13, wherein the determining of the skin type comprises:
based on rating configuration information on respective skin analysis items in which ratings are set to correspond to a plurality of threshold ranges for one or more skin analysis items, obtaining skin rating information corresponding to the first skin analysis information; and
determining a skin type corresponding to the obtained skin rating information.

15. A storage medium storing commands that, when executed by at least one processor, allow at least one processor to perform one or more operations of:
obtaining skin diagnosis information indicating a skin condition of a user;
determining a skin type of the user, based on the skin diagnosis information; and
displaying information on the determined skin type and content corresponding to the determined skin type on a display.
